(19) United States
(12) Patent Application Publication
(10) Pub. No.: US 2026/0226436 A1
(43) Pub. Date: Aug. 6, 2026

(54) COMPOSITIONS COMPRISING A SEQUENCE SPECIFIC ENDORIBONUCLEASE AND METHODS OF USE

(71) Applicant: ARCTICZYMES AS, Tromsø (NO)

(72) Inventors: Bernd Ketelsen Striberny, Kvaløya (NO); Ulli Rothweiler, Tromsø (NO)

(73) Assignee: ARCTICZYMES AS, Tromsø (NO)

(21) Appl. No.: 19/152,155

(22) PCT Filed: Feb. 7, 2024
(86) PCT No.: PCT/EP2024/053105

§ 371 Date: Jul. 30, 2025

(30) Foreign Application Priority Data
Feb. 7, 2023 (EP) 23155436.1

(51) Int. Cl. C12N 9/22; C12Q 1/6806; C12Q 1/6869

(52) U.S. Cl. CPC C12N 9/22; C12Q 1/6806; C12Q 1/6869

(57) ABSTRACT

The present disclosure provides compositions comprising sequence specific endoribonuclease and methods of their use in RNA analysis, RNA synthesis and fingerprinting of RNA molecules. In particular the present disclosure relates to compositions and samples comprising ToxN endoribonucleases that recognises and 5 cleaves single stranded RNA and optimal conditions for obtaining CN cleavage.

### FIELD OF THE INVENTION

The present disclosure provides compositions comprising sequence specific endoribonucleases and methods of their use in RNA analysis and RNA synthesis. In particular the present disclosure relates to compositions comprising ToxN endoribonucleases, subfamily ToxIN of the Type III Toxin-Antitoxin systems. ToxN endoribonucleases recognise and cleaves single stranded ribonucleic acid (RNA) molecules.

### INTRODUCTION

Endoribonucleases are a group of enzymes that cleaves internal phosphodiester bonds between adjacent nucleotides of RNA in either single-stranded RNAs or double-stranded RNAs depending on the enzyme. The endoribonuclease may be sequence specific (e.g. restriction endoribonucleases) or sequence independent.

A number of endoribonuclease enzymes are known, such as for example RNase H which is a family of sequence independent endoribonucleases that catalyse the cleavage of RNA in an RNA/DNA hybrid substrate.

Endoribonuclease enzymes have several applications within molecular biology research. For instance, removal of RNA in DNA extraction processes and recombinant protein purifications, cDNA synthesis, RNA fingerprinting, and detection of RNA modifications, e.g. 5′capping of mRNA.

Therapeutic RNA molecules such as mRNA molecules encoding antigens for vaccine production, represent an emerging class of drugs. Successful protein expression from transfected mRNA depends in addition to transfection efficiency also on mRNA stability and translation efficiency. 5′cap structure and 3′ poly(A) tail are important features for obtaining high translation efficiency. Efficient methods for determining 5′capping efficiency of mRNA or other RNA modifications is therefore highly desirable.

Analytical methods such as gel electrophoresis, ion-pair reverse-phase high-performance liquid chromatography (IP RP HPLC) or mass spectrometry (MS) are commonly used for analysing RNA modifications.

However, identification of capping completeness of long RNA molecules, i.e. RNA molecules longer than 5-10 ribonucleotides is problematic because capping results in only a small shift in molecular weight of approximately 600 Da, which equals roughly to one ribonucleotide. Such small shift in molecular weight prevents a direct down-stream gel-based or mass spectrometry (MS) analysis of long mRNA molecules due to poor resolution.

Alternative capping molecules result in different products exhibiting only small shifts in molecular weight: e.g. Cap-1 or Cap-0, 5′-triphosphate, 5′-diphosphate, unmethylated G-cap, reverse cap.

In order to overcome the problem with poor resolution of existing current analytical tools, RNA samples and in particular samples comprising long RNA molecules need to be cleaved into shorter fragments before further analysis.

Today's standards for cleavage of RNA are endoribonuclease-based cleavage with either enzymes with high cutting frequency, such as RNase I, RNase H-based methods, ribozyme-based methods, or DNAzyme-based approaches.

Endoribonucleases with high cutting frequency like RNase I cleaves single stranded RNA after each G. Such enzymes are not optimal for certain RNA analysis methods since they lead to a high degree of fragmentation.

RNase H which cleaves an RNA/DNA hybrid is dependent on specific DNA-hybridization, AU2016297778, and RNase H-based methods have problems with unspecific and incomplete cleavage of target RNA even if the DNA probe is correctly hybridized. Therefor RNase H-based methods requires optimalisation of conditions for each hybridized RNA-DNA oligonucleotide pair in order to achieve complete and specific digestion of the RNA.

The Csy4 endoribonuclease is dependent on a guide RNA for recognition and cleavage of its RNA target sequence.

Just recently, ribozymes have been described to be applicable for sequence-specific cleavage of mRNA as well, Vlatkovic et al., Ribozyme assays for quantifying the capping efficiency of in vitro transcribed mRNA, Pharmaceutics, 2022, vol. 14, no.2, p.328, and WO2015101416.

However, ribozymes rely of synthesis of ribonucleic acids and need to be used in 1-10-fold excess over the concentration of the substrate to be analysed. Ribozymes are catalytic RNA molecules and are thus more expensive to produce and also more challenging to work with due to lack of stability.

DNAzymes are DNA oligonucleotides with catalytic activity, similar to ribozymes. The most abundant class of deoxyribozymes are ribonucleases which catalyse cleavage of a ribonucleotide phosphodiester bond, Hengesbach, M. et al. Use of DNAzymes for site-specific analysis of ribonucleotide modification, RNA, 2008, vol. 14, no.1, p.180-187.

Despite the existence of endoribonucleases there is a continued need for providing further endoribonucleases which permit efficient and simplified methods for RNA analysis or RNA synthesis that overcome one or more of the disadvantages of the endoribonucleases and the methods of prior art.

The inventors have surprisingly and for the first time shown that sequence specific endoribonucleases of ToxIN subfamily from Type III toxin-antitoxin systems cleaves single stranded RNA specifically at its recognition site in the presence of particular concentrations of a monovalent salt, i.e. unspecific catalytic activity (also called star-activity”) of the enzyme is reduced in the absence or at low to moderate concentrations of monovalent salt.

The inventors have also for the first time determined that, surprisingly, the catalytic activity of endoribonucleases of the ToxIN subfamily of Type III toxin-antitoxin systems is inhibited by certain concentrations of divalent metal cations, which is in contrast to other endoribonucleases, i.e. the enzyme tolerate certain low concentrations of a divalent metal cation, but its enzyme activity is inhibited at higher concentrations.

The inventors have also for the first time determined that, surprisingly, the unspecific catalytic, i.e. its star-activity of the ToxN subfamily of endoribonucleases is reduced or absent at low concentrations of a divalent metal cation. The family of ToxN endoribonucleases are thus not dependent on divalent metal cations for their catalytic activity.

The inventors have also shown that it is not necessary to remove divalent metal cations from a reaction mixture comprising a ToxN endoribonuclease, rather the sequence specific catalytic activity may be obtained in the presence of a divalent metal cation chelator such as EDTA or EGTA. This is a great advantage as RNA samples are often stored in a buffer comprising EDTA to prevent degradation from RNases.

Such sequence specific catalytic activity at certain concentrations of monovalent salts and reduced unspecific cleavage of RNA in the absence or at low concentrations of divalent metal cations are not observed with other well-known endoribonucleases.

Divalent metal cations are known to stabilise RNA and protein three dimensional structures.

Without being bond by the theory, cleavage of RNA in the absence or at low concentrations of divalent metal cations may be more complete as such reaction conditions destabilizes the RNA three dimensional structures thereby improving the ToxN endoribonucleases' accessibility to its target site in the RNA molecule thereby decreasing the amount of enzyme needed for a complete digestion.

Further, as mentioned above, this class of endoribonucleases have the advantage that they digest single stranded RNA at specific sites without the need of a hybridized DNA probe or an RNA guide oligo. The family of ToxN endoribonucleases are also more efficient and more stable compared to catalytic nucleic acids such as ribozymes or DNAzymes which requires 1-10-fold excess over the concentration of the RNA substrate.

The above-mentioned advantages of the ToxN endoribonucleases compared to other well know endoribonucleases makes this family of endoribonucleases particularly useful in in vitro methods for analysing modification of RNA molecules such as efficiency of 5′capping and poly(A)-tail generation of synthetically transcribed RNA molecules. There is also provided herein the use of ToxN in methods for RNA fingerprinting and in methods for producing precursor RNA molecules from rolling circle transcription (RCT).

### SUMMARY OF THE INVENTION

In a first aspect there is provided a composition comprising an isolated ToxN endoribonuclease or a enzymatic active fragment thereof, wherein a concentration of a monovalent salts in the composition is ≤150 mM, such as about ≤100 mM and wherein the monovalent salt is preferably an alkali metal salt.

The ToxN endoribonucleases described herein comprises pFam domain PF13958.

In one embodiment of the first aspect the composition comprises a concentration of an alkali metal salt about ≤75 mM, such as about ≤55 mM, such as from about 20 mM to about 75 mM, such as from about 20 mM to about 55 mM.

In one embodiment of the first aspect the composition is a solution for application to a sample comprising at least one polyribonucleic acid (RNA) molecule.

In one embodiment of the first aspect said sample has a volume from about ≥0.1 μl.

In one embodiment of the first aspect said sample has a volume from 0.1 μl about to about 500 μl, preferably from about 0.1 μl to about 300 μl, preferably from about 0.1 μl to about 250 μl, preferably from about 0.1 μl to about 200 μl, more preferably from about 0.1 μl to about 150 μl, more preferably from about 0.1 μl to about 100 μl, more preferably from about 0.1 μl to about 75 μl, more preferably from about 0.1 μl to about 50 μl.

In one embodiment of the first aspect the monovalent salt of the composition or sample is an inorganic salt comprising alkali metal ions.

Thus, the monovalent salt is preferably an alkali metal salt.

In one embodiment of the first aspect the alkali metal ions of the salt are selected from Na+, K+, Li+, Rb+, Cs+ and Fr+ or any combinations thereof.

In one embodiment of the first aspect the alkali metal ions are selected from Na+, K+, Li+ and Rb+.

In one embodiment of the first aspect anions of the salts comprising alkali metals ions are preferably selected from fluorine (F), chlorine (Cl), bromine (Br), iodine (I), sulphates, phosphates or hydroxides or any suitable combinations thereof.

In one embodiment of the first aspect the alkali metal salt is selected from NaCl, KCl, Na2SO4, K2SO4, KOH, NaOH, Na-Phosphates, K-Phopshates or any suitable combinations.

In one embodiment of the first aspect the composition is essentially without divalent metal cations.

The divalent metal cations are preferably Mg²⁺ or Mn²⁺.

In one embodiment of the first aspect the composition is essentially without divalent metal cations, i.e. a concentration of divalent metal cations in the composition is about ≤3 mM, preferably about ≤2 mM, more preferably about ≤1 mM.

In one embodiment of the first aspect the composition is essentially without divalent metal cations, i.e. comprises ratio of concentration of a divalent metal cation to the concentration of divalent ion chelating agent in the composition providing that the concentration of free divalent metal cation present in the composition is about ≤3 mM, preferably about ≤2 mM, more preferably about ≤1 mM.

In one embodiment of the first aspect the composition comprises a concentration of a divalent ion chelating agent of about ≤10 mM.

The divalent ion chelator is preferably EDTA or EGTA.

In one embodiment of the first aspect the isolated ToxN enzyme is a ToxN enzyme from E. coli.

In one embodiment of the first aspect the isolated ToxN endoribonuclease or an enzymatically active fragment thereof comprises amino acid sequence of SEQ ID No.1 or comprising an amino acid sequence which is at least 30% identical to SEQ ID No.1.

In one embodiment of the first aspect the isolated ToxN endoribonuclease or an enzymatically active fragment thereof comprises amino acid sequence of SEQ ID No.1 or comprising an amino acid sequence which is at least 70% identical to SEQ ID No.1.

In one embodiment of the first aspect the isolated ToxN endoribonuclease or an enzymatically active fragment thereof comprises an amino acid sequence of SEQ ID No.1 or comprises an amino acid sequence which is at least 75%, 80%, 85%, 90%, 92%, 94%, 95%, 98% or 99% identical to SEQ ID No.1.

In one embodiment of the first aspect the isolated ToxN endoribonuclease or an enzymatically active fragment thereof comprises amino acid sequence of endoribonuclease having an amino acid sequence selected from:
SEQ ID No. 6 or an amino acid sequence which is at least 70% identical thereto,
SEQ ID No. 7 or an amino acid sequence which is at least 70% identical thereto,
SEQ ID No. 8 or an amino acid sequence which is at least 70% identical thereto, or
SEQ ID No. 13 or an amino acid sequence which is at least 70% identical thereto.

In one embodiment of the first aspect the ToxN endoribonuclease has an amino acid sequence which is at least 75%, preferably at least 80%, 85%, 90% or 95%, e.g. at least 98% or 99% or 99.5%, identical to SEQ ID Nos. 6, 7, 8 or 13.

In other embodiments of the first aspect the ToxN endoribonuclease consists of an amino acid sequence selected from the group consisting of SEQ ID Nos. 1, 6, 7, 8 and 13. Enzymatically active fragments thereof are also provided.

The isolated ToxN endoribonucleases disclosed herein is not in a complex with ToxI RNA.

In other embodiments of the first aspect the composition or sample comprises a ToxN endoribonuclease or an enzymatically active fragment thereof, said ToxN endoribonuclease comprises amino acid sequence of SEQ ID No.1 or comprising an amino acid sequence which is at least 30% identical such as 70% identical to SEQ ID No.1 wherein
concentration of monovalent salt in the composition or sample preferably about ≤100 mM, about ≤75 mM, about ≤55 mM, more preferably from about 20 mM to about 75 mM, more preferably from about 20 mM to about 55 mM.

In other embodiments of the first aspect the composition or sample comprises a ToxN endoribonuclease or an enzymatically active fragment thereof, said ToxN endoribonuclease comprises amino acid sequence of SEQ ID No.1 or comprising an amino acid sequence which is at least 30% identical such as at least 70% identical to SEQ ID No.1 wherein
concentration of monovalent salt in the composition or sample preferably about ≤100 mM, about ≤75 mM, about 55 mM, more preferably from about 20 mM to about 75 mM, more preferably from about 20 mM to about 55 mM;
   and wherein
the composition or sample is essentially without free divalent metal cations wherein the divalent metal cations are preferably Mg2+ or Mn2+ and wherein the divalent metal cations are provided as inorganic salts, such as MgCl2 or MnCl2.

In other embodiments of the first aspect the composition or sample comprises a ToxN endoribonuclease or an enzymatically active fragment thereof, said ToxN endoribonuclease comprises amino acid sequence of SEQ ID No.1 or comprising an amino acid sequence which is at least 30% identical such as at least 70% identical to SEQ ID No.1 wherein
concentration of monovalent salt in the composition or sample preferably about ≤100 mM, about ≤75 mM, about ≤55 mM, more preferably from about 20 mM to about 75 mM, more preferably from about 20 mM to about 55 mM;
   and wherein
concentration of free divalent metal cation is about ≤1 mM, the divalent metal cations are preferably Mg²⁺ or Mn²⁺ and wherein the divalent metal cations are provided as inorganic salts such as MgCl₂ or MnCl₂.

In other embodiments of the first aspect the composition or sample comprises a ToxN endoribonuclease or an enzymatically active fragment thereof, said ToxN endoribonuclease comprises amino acid sequence of SEQ ID No.1 or comprising an amino acid sequence which is at least 30% identical, such as at least 70% identical to SEQ ID No.1 wherein
concentration of monovalent salt in the composition or sample preferably about ≤100 mM, about ≤75 mM, about ≤55 mM, more preferably from about 20 mM to about 75 mM, more preferably from about 20 mM to about 55 mM;
   and wherein
the composition or sample comprises ratio of concentration of a divalent metal cation to the concentration of divalent ion chelating agent in the composition or sample such that the concentration of free divalent metal cation present in the sample or composition is about ≤1 mM and the concentration of divalent ion chelator is about ≤10 mM
the divalent metal cations are preferably Mg²⁺ or Mn²⁺ and provided as inorganic salts such as MgCl₂ or MnCl₂ and
the divalent ion chelating agent is preferably EDTA or EGTA.

In a second aspect there is provided a method of cleaving single stranded RNA molecules in a sample, wherein the method comprises the steps:
a. providing a sample comprising at least one single stranded RNA molecule comprising a cleavage site for a ToxN endoribonuclease; and
b. contacting a ToxN endoribonuclease or an enzymatically active fragment thereof with the at least one RNA molecule in said sample under conditions which permits cleavage of at least a portion said RNA molecule present in the sample, wherein concentration of a monovalent salt in the sample is about 150 mM, such as about 100 mM and wherein the monovalent salt is preferably an alkali metal salt.

In one embodiment of the second aspect said single stranded RNA molecule in step a) is a concatemer comprising multiple copies of precursors of either mRNA, siRNA, circular RNA precursors, microRNA or ribozyme and wherein the concatemeric RNA molecule comprising a cleavage site for a ToxN endoribonuclease between each copy of precursors of mRNA, siRNA, circular RNA, microRNA or ribozyme.

In one embodiment of the second aspect the cleavage step will typically be incubation which permits cleavage of at least a portion said RNA molecule present in the sample.

In one embodiment of the second aspect the composition and sample comprising ToxN is the composition and sample as described in the first aspect and embodiments thereof.

In one embodiment of the second aspect the incubation takes place at around 10° C. to around 50° C., such as around 10° C. to 30° C., preferably around 15° C.

In one embodiment of the second aspect the incubation the incubation time which permits cleavage of at least a portion said RNA molecule present in the sample is from about 1 minute to about 2 hours, such as from about 5 minutes to about 1.5 hours, such as from about 15 minutes to about 1 hour.

In a third aspect there is provided a method for preparing single stranded circular RNA molecules, wherein the method comprises the steps:
a. providing a sample comprising at least one single stranded RNA molecule wherein the RNA molecule comprises a cleavage site for a ToxN endoribonuclease;
b. contacting a ToxN endoribonuclease with the at least one single stranded RNA molecule under conditions that permit digestion of at least a portion of at least one RNA molecule present in the sample thereby producing at least one RNA molecule comprising a 3′-PO4 end and a 5′-OH end, wherein concentration of a monovalent salt in the sample is about ≤150 mM, such as about ≤100 mM and wherein the monovalent salt is preferably an alkali metal salt, and
c. contacting at least one cleaved RNA molecule with RtcB ligase under conditions which permit ligation thereby producing circular RNA.

In one embodiment of the third aspect the composition and sample comprising ToxN is the composition and sample as described in the first aspect and embodiments thereof.

In a further aspect there is provided a method of synthesizing siRNAs. The method comprising the steps:
a. providing a sample comprising at least one rolling circle transcribed concatemeric RNA molecule comprising cleavage sites for two different ToxN endoribonucleases, ToxN-A and ToxN-B, having different recognition sites, wherein the recognition sites for ToxN-B is situated between tandem repeats and recognition sequences for ToxN-A is situated within the tandem repeats;
b. contacting said sample with the ToxN-B endoribonuclease or an enzymatically active fragment thereof under conditions which permits cleavage of at least a portion of said RNA molecules present in the sample thereby producing single repeats that form a hairpin structure based on their sense-antisens sequence information; contacting the formed hairpin structures with a second ToxN-A enzyme that cleaves the RNA in the loop structure to form double stranded RNA molecules, wherein concentration of a monovalent salt in the sample is about ≤150 mM, such as about ≤100 mM and wherein the monovalent salt is preferably an alkali metal salt; and
c. the resulting overhangs containing parts of the ToxN recognition sites is removed by using a standard single-strand specific ribonuclease such as RNase T1 thereby producing double stranded siRNAs.

In one embodiment of the further aspect the composition and sample comprising ToxN is the composition and sample as described in the first aspect and embodiments thereof.

In a fourth aspect there is provided method for determining 5′-capping efficiency of a RNA molecule wherein the method comprises the steps:
a. providing a sample comprising at least one single stranded mRNA molecule comprising a cleavage site for a ToxN endoribonuclease in the 5′UTR of said mRNA;
b. contacting said sample from step a) with a ToxN endoribonuclease under conditions that permits cleavage of at least a portion of said single stranded RNA molecules to produce at least one 5′ terminal RNA fragment and at least one 3′ terminal RNA fragment; wherein concentration of a monovalent salt in the sample is about ≤150 mM, such as about ≤100 mM and wherein the monovalent salt is preferably an alkali metal salt; and
c. separating and detecting the RNA fragments from step b) and determine the presence of a 5′-capping modification at the 5′end of said 5′ terminal RNA fragments.

In one embodiment of the fourth aspect the 5′ terminal RNA fragment has a length from about 2 ribonucleotides to about 100 ribonucleotides, preferably 5 to 50, more preferably 5 to 10.

In one embodiment of the fourth aspect the composition and sample comprising ToxN is the composition and sample as described in the first aspect and embodiments thereof.

In a fifth aspect there is provided method for determining Poly(A) tail length distribution of a RNA molecule wherein the method comprises the steps:
a. providing a sample comprising at least one single stranded mRNA molecule comprising a cleavage site for a ToxN endoribonuclease upstream of the Poly(A) tail of said mRNA;
b. contacting said sample from step a) with a ToxN endoribonuclease under conditions that permits cleavage of at least a portion of said single stranded RNA molecules to produce at least one 5′ terminal RNA fragment and at least one 3′ terminal RNA fragment, wherein concentration of a monovalent salt in the sample is about 150 mM, such as about 100 mM and wherein the monovalent salt is preferably an alkali metal salt; and
c. separating and detecting the RNA fragments from step b) and determine the length of the Poly(A) tail at the 3′end of said 3′ terminal RNA fragments.

In one embodiment of the fifth aspect the composition and sample comprising ToxN is the composition and sample as described in the first aspect and embodiments thereof.

In a sixth aspect there is provided method of RNA fingerprinting, wherein the method comprises the steps:
a. providing a sample comprising a at least one single stranded RNA molecule with unknown sequence;
b. contacting said sample from step a) with a ToxN endoribonuclease under conditions that permit cleavage of at least a portion of said RNA molecules thereby obtaining a plurality of RNA fragments, wherein concentration of a monovalent salt in the sample is about 150 mM, such as about 100 mM and wherein the monovalent salt is preferably an alkali metal salt; and
c. separation and detection of the fragmented RNA molecule from step b, thereby obtaining a fingerprint of said RNA molecule with unknown sequence and compare the obtained fingerprint with fingerprints from RNA molecules with known sequence.

In one embodiment of the sixth aspect the composition and sample comprising ToxN is the composition and sample as described in the first aspect and embodiments thereof.

In a seventh aspect there is provided method for analysing a multivalent RNA composition, wherein the method comprises the steps:
a. providing a sample comprising a multivalent RNA composition comprising a first RNA species and a second RNA species comprising a cleavage site for a ToxN endoribonuclease in at least one position of said first RNA species and said second RNA species;
b. contacting said sample from step a) with a ToxN endoribonuclease under conditions that permits cleavage of at least a portion of said first RNA species and said second RNA species thereby releasing a plurality of first RNA fragments and second RNA fragments from the first and second RNA species, wherein concentration of a monovalent salt in the sample is about ≤150 mM, such as about ≤100 mM and wherein the monovalent salt is preferably an alkali metal salt; and
c. separation and detection the presence and/or amount of the released first RNA fragments and second RNA fragments.

In one embodiment of the sixth and seventh aspect the RNA molecule is selected from mRNA, RNA virus, an immunogenic RNA molecule, viroid, long non-coding RNA and ribozyme.

In one embodiment of the above methods the separation and detection step is based on different molecular weight, charge or length of the RNA fragments.

In one embodiment of the above methods the separation and detection step is selected from gel electrophoresis, capillary gel-electrophoresis (CGE), PAGE, high pressure liquid chromatography (HPLC), mass spectrometry (MS) or LC-MS, IP RP HPLC and LC-UV.

In one embodiment of the seventh aspect the composition and sample comprising ToxN is the composition and sample as described in the first aspect and embodiments thereof.

In an eight aspect there is provided kit comprising:
a. The composition according to the first aspect and embodiments therein; and
b. A second composition comprising a second enzyme selecting from a group consisting of an RNA polymerase, RNA ligase for ligating single stranded RNA molecules, a pyrophosphatase, a phosphatase, a kinase or any other nucleic acid or ribonucleic acid modifying enzymes and at least one further ToxN endoribonuclease with a different recognition site from the ToxN endoribonuclease of a).

### BRIEF DESCRIPTION OF DRAWINGS

FIG. 1: DNA sequence (SEQ ID NO: 3) ToxIN from E. coli encoding amino acid sequence SEQ ID NO: 1 (sequence with grey shade) also named ET-N1 herein; Antitoxin repeat (sequence within black frame) transcribed into ToxI (SEQ ID NO: 4); terminator (sequence within dotted line).

FIG. 2: Profiling ToxN (ET-N1) endoribonuclease activity: A: MW DNA ladder, B: 120 nM ToxN, C: 60 nM ToxN, D: 30 nM ToxN; E: 15 nM ToxN, F: 8 nM ToxN, G: 4 nM ToxN, H: 2 nM ToxN, I: 0 nM ToxN FIG. 3: Profiling ToxN (ET-N1) endoribonuclease activity: (1) 50 mM Tris-HCl pH 7.0, (2) 50 mM Tris-HCl pH 7.5, (3) 50 mM Tris-HCl pH 8.0, (4) 50 mM Tris-HCl pH 9.0; (A) MW DNA ladder; (B) control without ToxN; (C) 0 mM NaCl; (D) 25 mM NaCl; (E) 50 mM NaCl, (F) 75 mM NaCl; (G) 100 mM NaCl; (H) 125 mM NaCl; (I) 150 mM NaCl, (J) 175 mM NaCl.

FIG. 4a: Profiling ToxN (ET-N1) endoribonuclease activity: (A): MW DNA ladder, (B): w/o ToxN, (C): 0 mM MgCl₂, (D): 1 mM MgCl₂; (E): 2 mM MgCl₂, (F): 3 mM MgCl₂, (G): 4 mM MgCl₂, (H): 5 mM MgCl₂, (I): 6 mM MgCl₂, (J): 7 mM MgCl₂

FIG. 4b: Profiling ToxN (ET-N1) endoribonuclease activity: Lanes 1 to 6: 10 mM MgCl₂, 5 mM MgCl₂, 3 mM MgCl₂, 1 mM MgCl₂, 0 mM MgCl₂, No enzyme added; Lanes 7 to 12: 10 mM MnCl₂, 5 mM MnCl₂, 3 mM MnCl₂, 1 mM MnCl₂, 0 mM MnCl₂, No enzyme added.

FIG. 5: Profiling ToxN (ET-N1) endoribonuclease activity: (A): MW DNA ladder, (B): w/o ToxN, (C): ToxN, (D): 5 mM MgCl₂, (E): 5 mM EDTA, (F): 5 mM DTT 5 mM, (G): 5 mM MgCl₂, 5 mM EDTA, (H): 5 mM MgCl₂, 10 mM EDTA, (I): 5 mM MgCl₂, 5 mM EDTA, 5 mM DTT (J): 5 mM MgCl₂, 10 mM EDTA, 5 mM DTT

FIG. 6: Profiling ToxN (ET-N1) endoribonuclease activity: (A): MW DNA ladder, (B): w/o ToxN, (C): 5 min., (D): 10 min.; (E): 15 min., (F): 20 min., (G): 30 min., (H): 40 min., (I): 50 min., (J): 60 min.

FIG. 7: Profiling ToxN (ET-N1) endoribonuclease activity: (A): MW DNA ladder, (B): w/o ToxN, (C): 6° C., (D): 7° C.; (E): 10° C., (F): 13° C., (G): 17° C., (H): 22° C., (I): 27° C.

FIG. 8: Profiling ToxN (ET-N1) endoribonuclease activity: (A): MW DNA ladder, (B): w/o ToxN, (C): 25° C., (D): 25.2° C.; (E): 26° C., (F): 27° C., (G): 29° C., (H): 31° C., (I): 33° C., (J): 33.6° C., (K): 34° C., (L): 35° C., (M): 35.7° C., (N): 36° C.

FIG. 9a: illustrating the concept of determining 5′-capping efficiency of mRNA transcripts using a ToxN endoribonuclease.

FIG. 9b: depicts a capping analysis of an IVT (In Vitro Translated) construct of 40 bases made with the commercial kit “CleanCap”. The dark grey triangles show the 13 base (uncapped) and 14 base (capped) fragments after cleavage reaction with E. coli ToxN1. The addition bands marked with white triangles are caused by RNA initiation slippage. Lane 1: DNA Ladder: bands 50, 20, 15, 8, 6 bases; Lane 2: Capped IVT cut with E. coli ToxN1; Lane 3: uncapped IVT cut with E. coli ToxN1; Lane 4: capped IVT uncut; Lane 5: uncapped IVT uncut. The plasmid was linearized with HindIII. The 40 base nucleotide mRNA has the ToxN1 (ET-N1) cleavage site after 10 bases from the 5′end resulting in two fragments of 13 and 27 bases respectively. Successful capping can be seen by a band shift of the band to higher molecular weight.

FIG. 9c: depicts a capping analysis of an IVT (In Vitro Translated) construct. Gel picture (left): Lane 1: ET-N1 enzyme and an uncapped RNA oligo (GEM3Zf); Lane 2: ET-N1 enzyme and capped RNA oligo (GEM3Zf); lane 3: uncapped RNA oligo (GEM3Zf) w/o enzyme; lane 4: capped RNA oligo (GEM3Zf) w/o enzyme. The gel picture in the middle is an excerpt of the gel picture to the left. The diagrams to the right is a quantification of capped (<<, p 1) and uncapped (p2) mRNA fragments.

FIG. 9d: depicts a capping analysis of an IVT (In Vitro Translated) construct. Lane 1: ET-N1 enzyme and an uncapped RNA oligo (GEM3Zf); Lane 2: ET-N1 enzyme and capped RNA oligo (GEM3Zf); lane 3: uncapped RNA oligo (GEM3Zf) w/o enzyme; lane 4: capped RNA oligo (GEM3Zf) w/o enzyme.

FIG. 10a: illustrating the concept of mRNA-fingerprinting of an unknown virus strain in a sample. S1, S2, S3: RNA samples isolated from three different RNA viruses with unique distribution of recognition sites for ToxN. V: RNA sample isolated from unknown RNA virus. Analysis of fragment distribution by electrophoresis illustrates that the unknown virus (V) is a type (S2) virus.

FIG. 10b: shows fingerprinting of RNA by digestion of two 20 nucleotide RNA-FAM substrates simultaneously (MOD-UTR: GGGAA⬇AUAAGAGAGAAAAGA^{-FAM} and Dist1: GCCGAA⬇AUAGUGACCCUGCA^{-FAM}). The FAM labelled products differ by just one nucleotide resulting in product band of 15 and 14 nucleotides respectively. Lane 1: Uncut MOD-UTR; Lane 2: Cut MOD-UTR; Lanes 3-6: mixture of MOD-UTR and Dist1 in ratio 8:2, 6:4, 4:6, 2:8; Lane 7: Cut Dist1; Lane 8: Uncut Dist1. Only the product with the FAM label is visible.

FIG. 10c: depicts the ratio of the substrates in a mixture based on analysis of the band intensities of the FAM labelled oligonucleotides in FIG. 10b.

FIG. 11: illustrates the concept of RNA production by rolling circle transcription (RCT). (1) DNA plasmid for mRNA production, (2) single strand plasmid, (3) start of the RNA polymerase with a ToxN recognition site, (4) amplification of the RNA, once the circle is completed, (5) the RNA will continue to amplify RNA, (6) long chains of RNA with multiple copies of the mRNA of interest, (7) the RNA is cleaved by a ToxN endoribonuclease to yield multiple copies of the mRNA. This process can be simultaneous with RNA polymerase to constantly produce new mRNAs.

FIG. 12: illustrates rolling circle transcribed concatemeric RNA sequences to produce siRNAs. The transcribed sequence comprising ToxN recognition sequences for two different ToxN enzymes, ToxN-A and ToxN-B. The ToxN-B endoribonuclease will digest the RNA between pairs of concatemeric RNA sequences. Concatemeric RNA hybridize to form a hairpin structure. The ToxN-A enzyme cleaves RNA at its recognition site in the hairpin loop, the 5′-end and 3′end of the concatemeric RNA are digested with a single-strand specific RNase to remove the rest of the ToxN recognition sequences thereby producing a siRNA.

FIG. 13: depicts an IVT produced concatemer of the Mango aptamer digested with EcoToxN1 (ET-N1) at decreasing enzyme concentrations. Lane 1: No enzyme; Lane 2: 95 nM EcoToxN1; Lane 3: 47 nM; Lane 4: 23 nM; Lane 5: 12 nM; Lane 6: 6 nM; Lane 7: 2 nM; Lane 8: 1 nM.

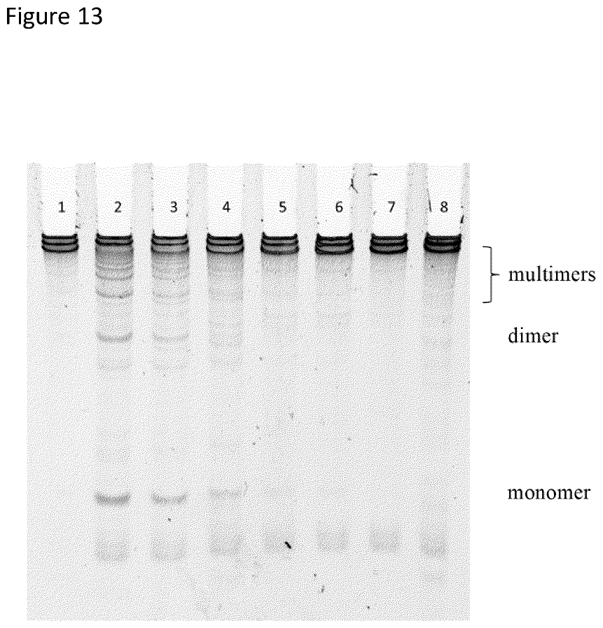

FIG. 14: profiling EcoToxN1 (ET-N1), EcoToxN5 (ET-N5) and BtuToxN (BT-N1) endoribonuclease activity. Lane 1: RNA oligo ladder; lane 2: ET-N1 and Q1 RNA oligo comprising ET-N1 recognition site; lane 3: Q1 RNA oligo w/o enzyme; lane 4: ET-N5 and RS2 RNA oligo comprising ET-N5 recognition site; lane 5: RS2 RNA oligo w/o enzyme; lane 6: ET-N5 and RS3 RNA oligo comprising ET-N5 recognition site; lane 7: RS3 RNA oligo w/o enzyme; lane 8: UTR-sequence with no recognition site for ET-N5; lane 9: UTR-sequence w/o enzyme; lane 10: RS3 RNA oligo with recognition site for BT-N1; lane 11: RS3 oligo w/o enzyme.

### DETAILED DESCRIPTION

Unless specifically defined herein, all technical and scientific terms used have the same meaning as commonly understood by a skilled artisan in the fields of genetics, biochemistry, molecular biology.

Where a numerical limit or range is stated herein, the endpoints are included. Also, all values and sub ranges within a numerical limit or range are specifically included as if explicitly written out.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will prevail.

In the following description, various examples and embodiments of the invention are set forth in order to provide the skilled person with a more thorough understanding of the invention. The specific details described in the context of the various embodiments and with reference to the attached drawings are not intended to be construed as limitations.

### Definitions

“A polyribonucleotide” refers to a polymeric form of ribonucleotides. In some aspects, a polyribonucleotide consists of ribonucleotides only. In other aspects, a polyribonucleotide comprises ribonucleotides, and one or more modified ribonucleotides, but does not include any deoxyribonucleotides. In other cases, a polyribonucleotide comprises ribonucleotides, and may comprise one or more modified ribonucleotides, and one or more deoxyribonucleotides (including modified deoxyribonucleotides).

The terms “ribonucleic acid”, RNA, “polyribonucleotide” are used interchangeably and refer to a polymeric form of ribonucleotides of any length.

“Isolated” refers to a protein or nucleic acid that, if naturally occurring, is in an environment different from that in which it may naturally occur. “Isolated” is meant to include proteins or nucleic acids that are within samples that are substantially enriched for the protein or nucleic acid of interest and/or in which the protein or nucleic acid of interest is partially or substantially purified. Where the protein or nucleic acid is not naturally occurring, “isolated” indicates the protein or nucleic acid has been separated from an environment in which it was made by either synthetic or recombinant means.

An endoribonuclease cleaves either a single- or double-stranded RNA. The endoribonucleases described in this patent application cleaves single-stranded RNA.

The terms “RNA digestion” or “RNA cleavage” are used interchangeably and refers to hydrolysis of phosphodiester bonds within a polyribonucleotide backbone in a sample.

The method of RNA digestion involves contacting a sample comprising single stranded RNA with a ToxN endoribonuclease under conditions which permit the digestion of at least a portion of the single stranded RNA present in the sample. The ToxN endoribonucleases are sequence specific and will completely digest a polyribonucleotide at their target site under sufficient time and reaction conditions that permit enzyme function. A digestion of at least a portion of the single stranded RNA present in the sample may numerically be expressed as at least 20%, 30%, 40%, 50%, 60%, 70%, 80%, 85%, 90%, 95% or 99%. Alternatively, 100% of the single stranded RNA present in the sample is digested, i.e. a complete digestion at their target site of all the RNA molecules present in the sample.

Endoribonucleases may cleave sequences which are similar to their target recognition sequence. This non-specific activity has been termed “star-activity” and should preferably be avoided. Star activity results from the recognition and cleavage of secondary cleavage sites in addition to a primary cleavage site. The secondary cleavage sites differ by one or more ribonucleotides from the primary recognition site. The star activity is characterised by the appearance of further bands in a gel electrophoresis which appear in addition to the band pattern of the complete digestion arising by specific cleavage.

The terms “contacting”, “contact”, “applying to”, “application”, “adding to” and “addition” have their ordinary meanings and are used interchangeably herein.

The present invention also covers the exact terms, features, values and ranges etc. in case these terms, features, values and ranges etc. are used in conjunction with terms such as about, around, generally, substantially, essentially, at least etc. (i.e., “about 3” shall also cover exactly 3 or “essentially free” shall also cover “free of/without”).

The term “at least one” should be understood as meaning “one or more”, and therefore includes both embodiments that include one or multiple components.

ToxN is a family of endoribonucleases from subfamily ToxIN of the Type III toxin-antitoxin (TA) systems. The toxin (ToxN) is a protein/enzyme, while the antitoxin (ToxI) consists of multiple repeats of RNA. The ToxN endoribonucleases described herein has a pFam domain PF13958. The toxic effects of the protein are neutralized by the specific antitoxin RNA sequence (ToxI). The toxin assembles with the individual antitoxin repeats into a cyclic complex in which the antitoxin forms a pseudoknot structure. This RNA antitoxin binds tightly to the toxin to form a hetero tetrameric or hetero hexameric cyclic, unique self-closing RNA-protein complexes, in which the toxin and antitoxin are arranged alternately in a 1:1 ratio, ToxIN complex. Identification and functional characterization of the structure of the ToxN-antitoxin (RNA) complex from E. coli is described in Manikandan, P. et al., Identification, functional characterization, assembly and structure of ToxIN type III toxin-antitoxin complex from E. coli, Nucleic acid research, 2022, vol. 50, no.3, p.1687-1700.

“ToxN” (endoribonuclease),” ToxI” (antitoxin RNA molecule/inhibitor of ToxN) and “ToxIN” (heteromeric protein/RNA complex of ToxN with ToxI).

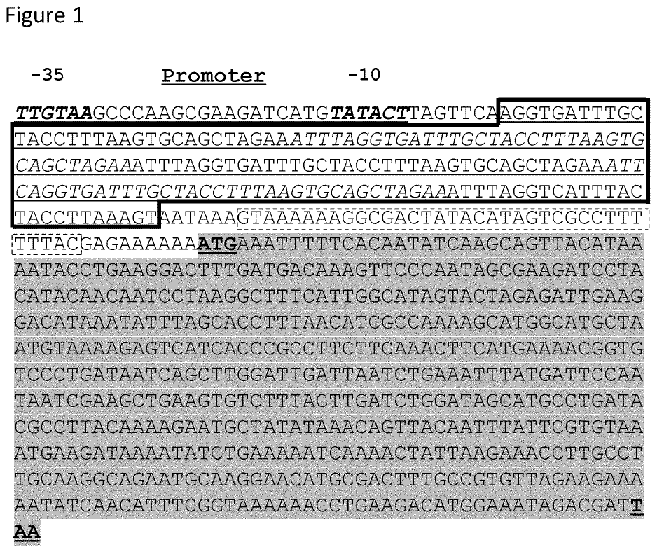

The DNA sequence encoding a ToxN endoribonuclease from E. coli, (NCBI accession number: PDB: 7D80_A) and which is neutralized by a RNA antitoxin ToxI and form together the inactive ToxIN complex is shown in FIG. 1 and SEQ ID NO: 3.

The number of ribonucleotides in the recognition sequence for the ToxN endoribonucleases may vary. The recognition sequence may be from 2 to 20 ribonucleotides. Preferably the recognition sequence is from 3 to 15 ribonucleotides, more preferably 4 to 10 ribonucleotides, 4 to 9 ribonucleotides or 4 to 6 ribonucleotides.

A non-limiting example of ToxN endoribonucleases comprising a pFam domain PF13958 and their recognition sequence are shown in table 1 below:

TABLE 1

| | | Type | | |
|---|---|---|---|---|
| | | III TA | | |
| NCBI acc. No./ | Homolog ToxN | sub | SEQ | Recognition |
| UniProt | endoribonucleases | family | ID NO | sequence in ssRNA |
| PDB: 7D80_A | ToxN E. coli | ToxN | 1 | 5′...GAA^{∧}AU..3′ |
| | (ET-N1) | | | |
| B8X8Z0 | ToxN | ToxN | 6 | 5′..GAA^{∧}AU...3′ |
| | Pectobacterium | | | |
| | atrosepticum | | | |
| Q3YN09 | ToxN Bacillus | ToxN | 7 | 5′..AAA^{∧}AAA..3′ |
| | thuringiensis subsp. | | | |
| | Kurstaki (BT-N1) | ToxN | | |
| Q9ZJ19.1 | ToxN (AbiQ), | | 8 | 5′...AA^{∧}AA..3′ |
| | Lactococcus lactis | | | |
| | subsp. lactis | | | |
| WP_208634907 | type III toxin- | ToxN | 13 | 5′..GAAA↓AAC..3′ |
| | antitoxin system | | | 5′.AAAA↓AUC..3′ |
| | ToxN/AbiQ family | | | |
| | toxin [Escherichia | | | |
| | albertii] (ET-N5) | | | |

The Type III toxin-antitoxin systems comprises at least three sub families of enzymes, the CptIN subfamily and the TenpIN subfamily, cf. Blower, T. R. et al., “identification and classification of bacterial Type III toxin-antitoxin systems encoding in chromosomal and plasmid genomes”, Nucleic acid research, 2012, Vol. 40, No. 13, p. 6158-6173.

The endoribonuclease disclosed by sequence CBK89509.1 does not belong to the ToxN subfamily according to the pFam classification. CBK89509.1 is classified as an alternative subfamily of the Type III TA systems, the CptIN sub-family.

| | | | | |
|---|---|---|---|---|
| CBK89509.1 | [Eubacterium] | CptN | 9 | 5′..GAA^{∧}AAG..3′ |
| | rectale | | | |

As mentioned above, the inventors have surprisingly and for the first time shown that a sequence specific endoribonuclease ToxN from Type III toxin-antitoxin systems cleaves single stranded RNA specifically at its recognition site in the presence of particular concentrations of a monovalent salt i.e. unspecific catalytic activity (also called star-activity”) of the enzyme is reduced or absent at particular concentrations of a monovalent salt.

The inventors have also for the first time determined that, surprisingly, the unspecific catalytic, i.e. its star-activity of ToxN is reduced or absent at low concentrations or without divalent metal cations, preferably Mg²⁺ or Mn²⁺, present in the composition or sample. Free divalent cation is to be understood as not bound to a divalent ion chelator such as EDTA or EGTA. The ToxN catalytic activity is also inhibited by concentrations of divalent cations above a certain concentration. Without being bound by the theory the divalent metal cations such as Mg²⁺ or Mn2+ present in the composition or sample may be bound to RNA thereby inhibiting the ToxN access to its target site.

The inventors have also shown that, surprisingly, it is not necessary to remove divalent metal cations from a composition or sample comprising a ToxN endoribonuclease, rather the sequence specific catalytic activity may be maintained by a divalent cation chelator such as EDTA or EGTA.

By “essentially free of divalent metal cations” in the context of a divalent ion chelating agent is meant that the ratio of the concentration of a divalent cation, preferably Mg2+ or Mn2+ to the concentration of divalent ion chelating agent, e.g. EDTA or EGTA, in the sample is from 3:1 to 1:10, such as (e.g. Mg²⁺ or Mn²⁺: EDTA or EGTA). Meaning that the ToxN enzyme tolerates a low concentration of a divalent metal cation present in the sample that is not bound to a divalent ion chelator without losing its catalytic activity.

This has the advantage that enzymes used in upstream applications and which requires a divalent metal cation such as Mg²⁺ or Mn²⁺ for optimal enzyme activity may be inactivated by the addition of a divalent ion chelator, e.g. EDTA or EGTA, and no subsequent purification step would be needed before the addition of ToxN, as ToxN catalytic activity and specificity is maintained when the composition or sample is essentially without free divalent cations, i.e. divalent cations not bound to an divalent ion chelator.

A further advantage is that EDTA or EGTA present in the sample may not need to be removed as the catalytic activity and specificity of the ToxN enzyme is not significantly affected by the presence of a divalent ion chelator, e.g. EDTA or EGTA.

The composition comprising isolated ToxN does not comprise a ToxIN complex.

The composition or sample comprising an isolated ToxN endoribonuclease or an enzymatically active fragment of the ToxN may not comprise monovalent salts.

The composition or sample comprising an isolated ToxN endoribonuclease or an enzymatically active fragment of the ToxN may not comprise free divalent metal cations.

The ToxN endoribonuclease may be a ToxN endoribonuclease from E. coli or an enzymatically active fragment thereof.

The expression “an enzymatically active fragment thereof” of the ToxN endoribonuclease is to be understood to mean a ToxN endoribonuclease wherein the catalytic activity of the endoribonuclease is maintained in the truncated form. Example 3 provide a suitable assay to measure endoribonuclease activity.

The ToxN endoribonuclease is preferably the ToxN with an amino acid sequence of SEQ ID NO: 1 (NCBI Acc. No.: PDB: 7D8O_A) or an amino acid sequence which is at least about 70% identical to SEQ ID NO: 1. Example of sequences with least 70% sequence identity to SEQ ID NO: 1 is the sequence with SEQ ID NO: 6. SEQ ID NO: 6 has 81.4% sequence identity to SEQ ID NO: 1.

The ToxN endoribonuclease may be ToxN with an amino acid sequence of SEQ ID NO: 1 (NCBI Acc. No.: PDB: 7D8O_A) or an amino acid sequence which is at least about 30% identical to SEQ ID NO: 1. Examples of sequences with at least 30% sequence identity to SEQ ID NO: 1 are the sequences with SEQ ID NO: 7 (30.95% identity), SEQ ID NO: 8 (32.5% identity) and SEQ ID NO: 13 (40.88% identity).

The ToxN endoribonuclease may be a ToxN endoribonuclease or an enzymatically active fragment thereof wherein the ToxN comprises an amino acid sequence which is at least 30%, 35%, 40%, 45%, 50%, 55%, 60%, 65%, 70%, 75%, 80%, 85%, 90%, 92%, 94%, 95%, 98% or 99% identical to SEQ ID No.1.

By “at least about 30%” it is meant that the sequence identity may be at least 29%, 29.5% or 29.9% to SEQ ID No.1.

By “at least about 70%” it is meant that the sequence identity may be at least 69%, 69.5% or 69.9% to SEQ ID No.1.

The ToxN endoribonuclease may consists of the amino acid sequence of SEQ ID No 1. Enzymatically active fragments thereof are also provided.

An endoribonuclease having an amino acid sequence which is at least 30% identical to SEQ ID No.1 may be obtained from a prokaryotic organism

An endoribonuclease having an amino acid sequence which is at least 70% identical to SEQ ID No.1 may be obtained from a prokaryotic organism.

Thus, in another aspect there is provided a composition comprising a ToxN endoribonuclease or an enzymatically active fragment thereof, said ToxN endoribonuclease having an amino acid sequence selected from
(a) SEQ ID No. 6 or an amino acid sequence which is at least 70% identical thereto,
(b) SEQ ID No. 7 or an amino acid sequence which is at least 70% identical thereto,
(c) SEQ ID No. 8 or an amino acid sequence which is at least 70% identical thereto, or
(d) SEQ ID No. 13 or an amino acid sequence which is at least 70% identical thereto.

In one embodiments, the ToxN endoribonuclease has an amino acid sequence which is at least 75%, preferably at least 80%, 85%, 90% or 95%, e.g. at least 98% or 99% or 99.5%, identical to SEQ ID Nos. 1, 6, 7, 8 or 13. In other embodiments the ToxN endoribonuclease consists of an amino acid sequence selected from the group consisting of SEQ ID Nos. 1, 6, 7, 8 and 13. Enzymatically active fragments thereof are also provided.

Variants of SEQ ID No. 1, 6, 7, 8, 13 include amino acid sequences in which one or more amino acids of said amino acid sequences have undergone conservative substitutions. Preferably such substitutions are silent substitutions in that the modified form of the ToxN endoribonucleases of the invention have the same enzymatic activity as the unmodified form.

As used herein, when referring to “sequence identity” of proteins, an amino acid sequence having at least x % identity to a second amino acid sequence means that x % represents the number of amino acid residues in the first sequence which are identical to their matched amino acid residues of the second sequence when both sequences are optimally aligned via a global alignment, relative to the total length of the second amino acid sequence. Both sequences are optimally aligned when x is maximum by using Clustal Omega with default settings, the multiple protein sequence alignment tool from EMBL-EBI.

Preferably the composition is a solution, preferably an aqueous solution. The term “solution” as used herein means a liquid mixture in which one or more minor components (solutes) are uniformly distributed within a major component (solvent).

Typically, the minor component (solute) of a solution is soluble in the major component (solvent).

Preferably, the major component, i.e. solvent, i.e. liquid phase, is water. Preferably, the solution comprises water. The solutions of the present invention comprise at least a ToxN endoribonuclease or an enzymatically active fragment thereof as a minor component.

In a preferred embodiment, the solution is a reagent for application to a sample comprising one or more RNA molecules. Such a reagent is applied to a sample in order for the ToxN endoribonuclease in said reagent to digest said one or more ribonucleotides present in the sample. Preferably, the sample comprises multiple ribonucleotides. In this embodiment, the solution comprises a ToxN endoribonuclease or enzymatically active fragment thereof.

The term “sample” refers to a composition comprising single stranded RNA molecules.

The composition also comprises a buffer. Suitable buffers are well known in the art and any such buffer may be used. It would be within the competencies of a skilled person in the art to identify a suitable buffer.

The buffer has a buffering range of about pH 5.5 to about pH 9, preferably about pH 6.5 to about pH 9, preferably about pH 7 to about pH 9, more preferably about pH 7 to about pH 8.5.

The buffer may be Tris, HEPES or phosphate buffer. Preferably, the buffer is present in the composition or sample at a concentration of 1 mM to 200 mM, preferably 10 mM to 200 mM, preferably 20 mM to 150 mM, more preferably 25 mM to 100 mM.

If present, preferably Tris-HCl is present at a concentration of 25 mM to 150 mM, more preferably 40 mM to 100 mM, more preferably about 50 mM.

The samples may have a volume of >0.1 μl. Preferably the samples of the invention have a volume of from about 0.1 μl to about 500 μl, such as from about 0.1 μl to about 300 μl, such as from about 0.1 μl to about 250 μl, such as from about 0.1 μl to about 200 μl, such as from about 0.1 μl to about 150 μl, such as from about 0.1 μl to about 100 μl, such as from about 0.1 μl to about 75 μl, such as from about 0.1 μl to about 50 μl.

The skilled person is able to determine the appropriate concentration of the enzyme to include in the sample and the reaction mixture in order to obtain digestion of preferably all RNA molecules in the sample and at the same time avoiding unspecific digestion and star activity.

The reaction mixture may comprise further components that may be present due to having been added at an earlier stage of the workflow and tolerable in the reaction assay, e.g. DTT, nucleotides, S-adenosylmethionine (SAM) or other enzymes. DTT is a reducing agent that stabilizes disulphide bonds within enzymes and thereby stabilizing the enzyme structure.

As described above the composition or sample comprising ToxN endoribonuclease or an enzymatically active fragment thereof comprises preferably a specific concentration of a monovalent salt.

The monovalent salt present in the composition or sample may selected from inorganic salts comprising alkali metal ions wherein the alkali metal ions are selected from Na+, K+, Li+, Rb+, Cs+ and Fr+ or any combinations thereof.

Thus, preferably the monovalent salt is an alkali metal salt.

Preferably the alkali metal ions of the salt are selected from Na+, K+, Li+ and Rb+.

The anions of the salts comprising alkali metals ions are preferably selected from fluorine (F), chlorine (Cl), bromine (Br), iodine (I), sulphates, phosphates or hydroxides or any suitable combinations thereof.

Preferably the alkali metal salts are NaCl, KCl, Na2SO4, K2SO4, KOH, NaOH, Na-Phosphates, K-Phopshates or any suitable combinations.

Thus, preferably the composition or samples comprising a particular concentration of a monovalent salt is referred herein to a composition or sample that comprises a concentration of monovalent salt ≤150 mM.

The term “about ≤X mM” is equivalent to from 0 to about X mM”.

The composition or sample may not comprise a monovalent salt.

Thus, in one aspect the composition or sample comprising a ToxN endoribonuclease or an enzymatic active fragment thereof comprises a concentration of alkali metal salt in the composition or sample ≤150 mM, about ≤100 mM, about 75 mM, about ≤55 mM, from about 20 mM to about 75 mM, from about 20 mM to about 55 mM.

As described above the compositions or samples comprising a ToxN endoribonuclease or an enzymatic active fragment thereof are essentially without free divalent metal cations, i.e. the composition or sample may comprise low concentration of a divalent metal cation. Free divalent metal cations are referred herein to divalent metal cations not bound to a divalent cation chelator such as EDTA or EGTA.

Thus, the compositions or samples may comprise a concentration of free divalent metal cation about ≤3 mM.

The composition or samples may be without free divalent metal cations.

The free divalent cations are preferably selected from Mg²⁺ and Mn²⁺.

The composition or samples may be without free Mg²⁺.

The composition or samples may be without free Mn²⁺.

Preferably, the concentration of free Mg²⁺ and/or free Mn²⁺ in the compositions and samples are about ≤2 mM, more preferably about ≤1 mM.

The composition or sample comprising a ToxN endoribonuclease or an enzymatic active fragment thereof may comprise a concentration of free Mg²⁺ and/or Mn²⁺ in the composition or sample in the range from 0 to about 1 mM, from about 1 μM to about 1 mM, from about 1 μM to about 0.9 mM, from about 1 μM to about 0.8 mM, from about 1 μM to about 0.7 mM, from about 1 μM to about 0.6 mM, from about 1 μM to about 0.5 mM.

The composition or sample comprising a ToxN endoribonuclease or an enzymatic active fragment thereof may comprise a ratio of concentration of a divalent metal cation to a concentration of a divalent ion chelating agent in the composition or sample such that the max concentration of free divalent cation in the composition or sample, i.e. not bound to a divalent ion chelator is not above 3 mM, preferably not above 2 mM and more preferably not above 1 mM.

The composition or sample comprising a ToxN endoribonuclease or an enzymatic active fragment thereof may comprise a concentration of free Mg²⁺ in the composition or sample in the range from 0 to about 3 mM.

The composition or sample comprising a ToxN endoribonuclease or an enzymatic active fragment thereof may comprise a concentration of free Mn²⁺ in the composition or sample in the range from 0 to about 1 mM.

The composition or sample comprising a ToxN endoribonuclease or an enzymatic active fragment thereof may comprise a concentration of a divalent ion chelator of about ≤10 mM.

The divalent ion chelator is preferably EDTA or EGTA.

For solubility reasons the compositions and samples comprising divalent metal cations are preferably added as a divalent salt. A divalent salt is a salt in which at least one of the counter ions is divalent, e.g. MgCl₂ or MnCl₂. The salts are preferably inorganic.

An inorganic salt is a salt in which neither of the counter ions comprises carbon.

The mono- or divalent salts are preferably inorganic salts.

Preferably the compositions and samples comprise monovalent salts and preferably also monovalent anions which are counterions. The preferred concentrations of monovalent salts disclosed herein are, inherently, the preferred concentrations of monovalent counterions, and vice versa.

Thus, there is provided herein a composition or a sample comprising a ToxN endoribonuclease or an enzymatically active fragment thereof, said ToxN endoribonuclease comprises amino acid sequence of SEQ ID No.1 or comprising an amino acid sequence which is at least 30% identical such as 70% identical to SEQ ID No.1 wherein
concentration of monovalent salt in the composition or sample is about ≤100 mM, about ≤75 mM, about ≤55 mM, from about 20 mM to about 75 mM, preferably from about 20 mM to about 55 mM.

It is also provided a composition or a sample comprising a ToxN endoribonuclease or an enzymatically active fragment thereof, said ToxN endoribonuclease comprises amino acid sequence of SEQ ID No.1 or comprising an amino acid sequence which is at least 70% identical to SEQ ID No.1 wherein
concentration of monovalent salt in the composition or sample is about ≤100 mM, about ≤75 mM, about ≤55 mM, preferably from about 20 mM to about 75 mM, preferably from about 20 mM to about 55 mM
   and wherein
the composition or sample is essentially without free divalent metal cations wherein the divalent metal cations are preferably Mg2+ or Mn2+ and wherein the divalent metal cations are provided as inorganic salts, such as MgCl2 or MnCl2.

There is also provided a composition or a sample wherein the ToxN endoribonuclease or an enzymatically active fragment thereof, said ToxN endoribonuclease comprises amino acid sequence of SEQ ID No.1 or comprising an amino acid sequence which is at least 30% identical such as at least 70% identical to SEQ ID No.1 wherein
concentration of monovalent salt in the composition or sample is about ≤100 mM, about 75 mM, about 55 mM, preferably from about 20 mM to about 75 mM, preferably from about 20 mM to about 55 mM
   and wherein
concentration of free divalent metal cation is about ≤1 mM, the divalent metal cations are preferably Mg²⁺ or Mn²⁺ and wherein the divalent metal cations are provided as inorganic salts such as MgCl₂ or MnCl₂.

There is also provided a composition or a sample wherein the ToxN endoribonuclease or an enzymatically active fragment thereof, said ToxN endoribonuclease comprises amino acid sequence of SEQ ID No.1 or comprising an amino acid sequence which is at least 30% identical, such as at least 70% identical to SEQ ID No.1 wherein
concentration of monovalent salt in the composition or sample is about ≤100 mM, about ≤75 mM, about ≤55 mM, preferably from about 20 mM to about 75 mM, preferably from about 20 mM to about 55 mM.
   and wherein
the composition or sample comprises ratio of concentration of a divalent metal cation to the concentration of divalent ion chelating agent in the composition or sample such that the concentration of free divalent metal cation present in the sample or composition is about ≤1 mM and the concentration of divalent ion chelator is about ≤10 mM
the divalent metal cations are preferably Mg²⁺ or Mn²⁺ and provided as inorganic salts such as MgCl₂ or MnCl₂ and
the divalent ion chelating agent is preferably EDTA or EGTA.

As explained above free Mg²⁺ or Mn²⁺ in the sample is denoted herein as not bound to EDTA or EGTA.

Thus for example the ratio of concentration of a divalent metal cation to the concentration of divalent ion chelating agent in the composition or sample may be for example 2 mM: 1 mM, 5 mM: 5 mM, 5 mM: 10 mM, 10 mM: 10 mM or any other alternative ratio combinations providing that the concentration of free divalent metal cation in the composition or sample is about ≤1 mM and the concentration of divalent ion chelator is preferably about ≤10 mM.

### Preparation of the ToxN Endoribonuclease of the Present Invention

A method of preparing ToxN endoribonuclease is described in Manikandan, P. et al., Identification, functional characterization, assembly and structure of ToxIN type III toxin-antitoxin complex from E. coli, Nucleic acid research, 2022, vol. 50, no.3, p.1687-1700 and further described in Example 1.

The ToxN endoribonuclease and the enzymatically active fragments thereof or nucleic acid molecules encoding the endoribonuclease may be isolated from a natural source such as a bacteria, for example E. coli, Pectobacterium atrosepticum, Bacillus thuringiensis subsp. Kurstaki, Lactococcus lactis subsp. lactis or [Eubacterium] rectale.

Alternatively, the enzyme may be produced recombinantly in a host cell and isolated and purified therefrom. Wherein the host cell is not, or not from an organism which naturally express the gene encoding the ToxN endoribonuclease, i.e. the host cell is a heterologous host cell such as a yeast cell, an insect cell, a human cell line or a bacterial cell, preferably E. coli.

Nucleic acid sequences encoding the ToxN endoribonuclease or enzymatic active fragments thereof according to the present invention may be amplified using PCR from genomic DNA, isolated as a cDNA or may be ordered by a commercial supplier such as GENEWIZ, GeneArt from Thermo Fisher Scientific or Genscript.

As described above the nucleic acid sequence encoding the ToxN endoribonuclease or enzymatic active fragments thereof may be codon optimized for increased protein production in a heterologous host cell. A variety of software programs for help with codon-optimization are well known in the art. CodonW is an example of an open source software program that may be used. Preferably the GeneOptimizer algorithm described by Raab, D., Graf, M., Notka, F., Schödl, T., & Wagner, R. (2010). The GeneOptimizer Algorithm: Using a sliding window approach to cope with the vast sequence space in multiparameter DNA sequence optimization. Systems and Synthetic Biology, 4(3), 215-225, is used for generating a codon optimized DNA sequence for expression of the ToxN endoribonuclease in a E. coli host cell.

There are various available molecular techniques for expression of proteins from DNA sequences by heterologous expression in various host cell systems using well known recombinant gene expression systems. For example, the nucleic acid molecule encoding a ToxN endoribonuclease or encoding an enzymatic active fragment thereof may be inserted in a suitable expression vector comprising necessary transcriptional and translational elements for expression that are appropriate for the chosen host cell. Example of commonly used expression vectors are plasmids or viruses.

To ensure a reliable transcription of the gene of interest. The expression vector may comprise a strong promoter, bacteriophage T5 and T7 are examples of strong promoters for expression in E. coli. The promoter may be regulated by comprising chemical switches. Example of inducible promoters for use in E. coli is the commonly used lac promoter induced by Isoropyl-beta-D-thiogalactoside (IPTG) (Hansen LH, Knudsen S, Sorensen SJ, “The effect of the lacY gene on the induction of IPTG inducible promoters, studied in Escherichia coli and Pseudomonas fluorescens”, Curr. Microbiol. 1998, 36 (6): 341-7) or the XylS/Pm expression cassette comprising a promoter that is inducible with toluic acid (Gawin, A. et al., The XylS/Pm regulator/promoter system and its use in fundamental studies of bacterial gene expression, recombinant protein production and metabolic engineering, Microb. Biotechnol., 2017, Vol. 10, No. 4, p 702-718). The XylS/Pm regulator/promoter system originating from the Pseudomonas putida is widely used for regulated low- and high-level recombinant expression of genes and gene clusters in E. coli and other bacteria.

A further aspect of the invention is a method of expression of a ToxN endoribonuclease or an enzymatic active fragment thereof as described above in a suitable heterologous cell. The host cell may be a bacterium or a yeast cell. Preferably the expression of the enzyme is in a bacterial host cell, more preferably E. coli, BL21 (DE3) cells.

Transformation of the above described expression vector comprising the ToxN endoribonuclease may be performed by methods well known to a skilled person, e.g. by using chemically competent cells.

As described above, the ToxN endoribonuclease may be synthesized using recombinant DNA technology. Alternatively, the endoribonuclease may be produced using a cell-free expression system or chemical synthesis of a ToxN endoribonuclease.

A ToxN endoribonuclease enzyme comprising a signaling peptide for secretion into cell culture media may be isolated and purified from the host cell culture media using any technique known in the art and well described in literature. Examples of such techniques or any combination may include precipitation, ultrafiltration, different chromatographic techniques e.g. size-exclusion chromatography, immobilized metal affinity column chromatography and/or immunoadsorption chromatography.

A ToxN endoribonuclease produced intracellularly may be isolated and purified also using techniques well known to a skilled person. Examples of methods for preparation of cell lysates from E. coli cells are homogenization, sonication or enzymatic lysis using lysozyme. After the ToxN endoribonuclease is released from the lysed cells the enzyme may be subject to any method of purification for example size-exclusion chromatography, immobilized metal affinity column chromatography and/or immunoadsorption chromatography.

The ToxN endoribonuclease may comprise a C-terminal or N-terminal His-tag to ease isolation, purification and/or identification of the enzyme. An N-terminal poly-histidine tagged ToxN endoribonuclease is depicted in SEQ ID No. 2.

The purified ToxN endoribonuclease or an enzymatic active fragment thereof may finally be stored in a buffer.

The purified ToxN endoribonuclease enzyme or an enzymatic active fragment thereof may finally be stored in a suitable buffer. Suitable buffers for storing the ToxN endoribonuclease is known to a skilled person.

It has been found that ToxN enzymes are particularly stable if stored in a buffer comprising from about 100 mM monovalent salt to about 500 mM. The monovalent salt may be selected from NaCl?

Preferably the alkali metal ions of the salt are selected from Na+, K+, Li+ and Rb+.

The anions of the salts comprising alkali metals ions are preferably selected from fluorine (F), chlorine (Cl), bromine (Br), iodine (I), sulphates, phosphates or hydroxides or any suitable combinations thereof.

Preferably the alkali metal salts are NaCl, KCl, Na2SO4, K2SO4, KOH, NaOH, Na-Phosphates, K-Phopshates or any suitable combinations.

### Kits Comprising ToxN Endoribonucleases

The compositions and samples of the present invention comprises an endoribonuclease that is not inhibited by the presence of a divalent ion chelator, which is often added to enzyme reactions to stop the catalytic activity of an enzyme. The ToxN endoribonucleases therefore have advantageous utility in various molecular biology methods, which involve prior use of other enzymes. Such methods are discussed in more detail below. Thus, in a further aspect there is provided a kit comprising:
i) a composition comprising a ToxN endoribonuclease as defined above; and
ii) a second composition comprising a second enzyme selecting from a group consisting of a RNA polymerase, a RNA ligase for ligating single stranded RNA molecules, a pyrophosphatase, a phosphatase, a kinase or any other nucleic acid or ribonucleic acid modifying enzymes and at least one further ToxN endoribonuclease with a different recognition site from the ToxN endoribonuclease of a).

The second enzyme may be another ToxN endoribonuclease having a different recognition site compared to the ToxN enzyme as mentioned in i).

Kits comprising a ToxN endoribonuclease may comprise a suitable buffer for carry out digestion of RNA.

Suitable RNA digestion buffers and reaction conditions for carry out cleavage of RNA is described in detail above.

### Method of Cleaving Single Stranded RNA

The enzymatic activity of the ToxN endoribonucleases makes such enzymes especially suited for use in methods that involves cleavage of single stranded RNA.

The different ToxN endoribonucleases cleave single stranded RNA molecules at different recognition sites.

Thus, in one aspect the composition comprising a ToxN endoribonuclease may be a solution for application to a sample wherein the sample comprising at least one isolated single stranded RNA molecule comprising at least one cleavage site for a ToxN endoribonuclease.

In a further aspect there is provided a method of cleaving single stranded RNA molecules in a sample, wherein the method comprises the steps:
a. providing a sample comprising at least one single stranded RNA molecule comprising at least one cleavage site for a ToxN endoribonuclease; and
b. contacting a ToxN endoribonuclease or an enzymatically active fragment thereof with said sample under conditions which permits cleavage of at least a portion of said RNA molecules present in the sample.

Preferably the ToxN endoribonucleases are the ToxN endoribonucleases as defined above.

Suitable RNA digestion buffers and reaction conditions for carry out cleavage of RNA is described in detail above.

The step of digestion will typically be incubation and is described above and in the examples. Suitable incubation comprises incubation at around 10° C. to around 50° C., such as around 10° C. to 30° C., preferably around 15° C. for 1 minute to about 2 hours, such as from about 5 minutes to about 1.5 hours, such as from about 15 minutes to about 1 hour.

The single stranded RNA molecule in the sample may be a concatemer comprising multiple copies of precursors of either mRNA, siRNA, circular RNA, precursors, microRNA or ribozyme and wherein the concatemeric RNA molecule comprises a cleavage site for a ToxN endoribonuclease between each copy of precursors of mRNA, siRNA, circular RNA, microRNA or ribozyme.

### Method for Preparing Circular RNA Molecules

The nature of the RNA 5′end and 3′end produced by ToxN can be exploited through subjecting the RNA to a RtcB Ligase reaction which ligates 3′-PO₄ ends with 5′-OH ends of RNA. By this reaction the RNA becomes circular. The mechanism of making circular RNA by the use of RtcB ligases is described in Tanaka, N. et al., Novel mechanism of RNA repair by RtcB via sequential 2′, 3′-cyclic phosphodiesterase and 3′-phosphate/5′-hydroxyl ligation reactions, J Biol Chem., 2011, vol. 286, no.50, p.43134-43143.

In a further aspect there is provided a method for preparing single stranded circular RNA molecules present in a sample wherein the RNA molecules comprise a cleavage site for a ToxN endoribonuclease; contacting a ToxN endoribonuclease with said RNA molecule under conditions that permit digestion of at least a portion of the RNA molecules present in the sample thereby producing RNA molecules comprising 3′-PO₄ ends and 5′-OH ends; contacting the digested RNA molecules with RtcB ligase under conditions which permits ligation thereby producing circular RNA.

### Method of Preparing Multiple Copies of RNAs by Rolling Circle Transcription (RCT)

Rolling circle transcription (RCT), using small circular single-stranded DNA as the template, has been well investigated in the past two decades. Interestingly, transcription via the rolling circle mechanism is achieved by using the T7 RNA polymerase but may happen in absence of the specific canonical promoters and generate transcripts that are tandemly repeated sequences complementary to the circular template. In the literature the use of RNase H (Wang et al., Preparation of small RNAs using rolling circle transcription and site-specific RNA disconnection, Molecular Therapy-Nucleic Acids, 2015, e215) or ribozymes (WO2020023741) are reported.

By adapting the method to the use of site specific ToxN endoribonucleases the method is simplified and no longer requires RNase H and a helper DNA fragment or the use of synthetic or in-the-transcript encoded ribozymes.

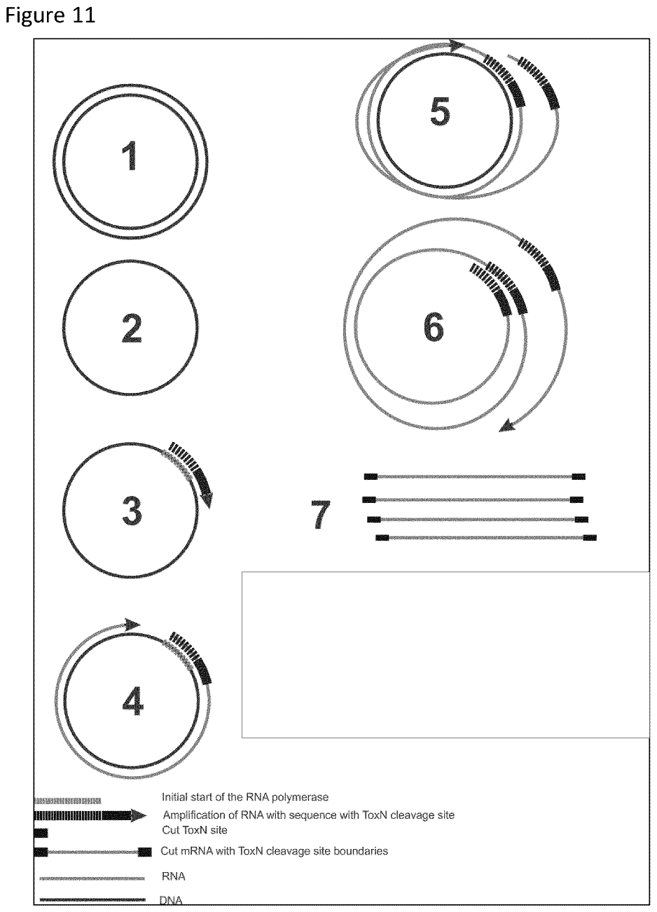

Thus, in a further aspect there is provided a method for synthesizing RNA by rolling circle transcription (RCT), as outlined in FIG. 11. The method comprises the steps:
providing a single stranded DNA plasmid comprising a sequence encoding an RNA recognition site for a ToxN endoribonuclease optionally in proximity of a recognition site (also called promoter) for an RNA polymerase;
amplification of the RNA;
once the circle is completed, the RNA polymerase will continue to amplify RNA into long chains with multiple copies of the RNA of interest;
the RNA is cleaved by ToxN to yield multiple copies of the RNA. This process can be simultaneous with RNA polymerase to constantly produce new RNAs.

Preferably the ToxN endoribonucleases are the ToxN endoribonucleases as defined above.

Suitable RNA digestion buffers and reaction conditions for carry out cleavage of RNA is described in detail above.

Method of Preparing Double Stranded siRNAs Small interfering RNA (siRNA), sometimes known as short interfering RNA or silencing RNA, is a class of double-stranded RNA typically 20-24, normally around 21 base pairs in length, i.e. similar to miRNA. siRNAs operate within the RNA interference (RNAi) pathway and it interferes with the expression of specific genes with complementary nucleotide sequences by degrading mRNA after transcription, thereby preventing translation.

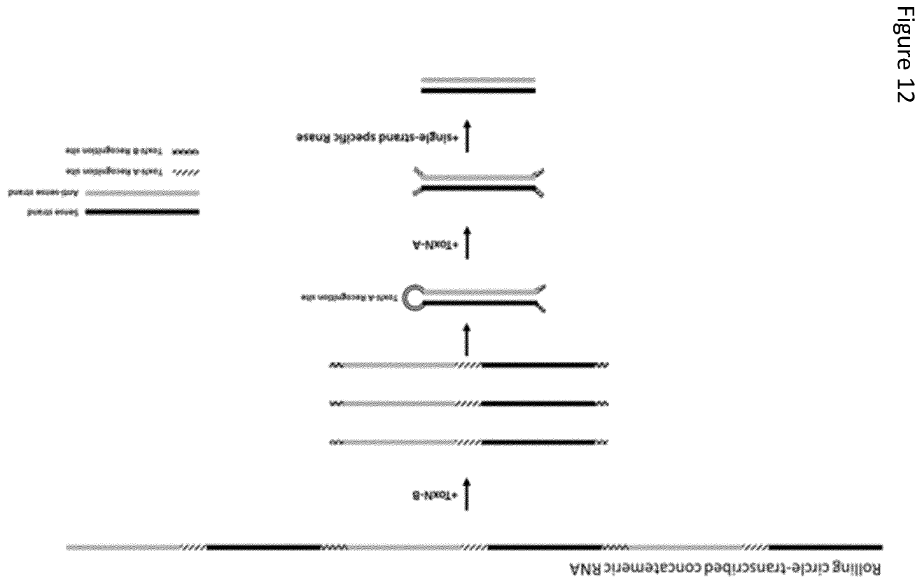

In a further aspect there is provided a method of synthesizing siRNAs. The method comprising the steps:
a. providing a sample comprising at least one rolling circle transcribed concatemeric RNA molecule comprising cleavage sites for two different ToxN endoribonucleases, ToxN-A and ToxN-B, having different recognition sites, wherein the recognition sites for ToxN-B is situated between tandem repeats and recognition sequences for ToxN-A is situated within the tandem repeats;
b. contacting said sample with the ToxN-B endoribonuclease or an enzymatically active fragment thereof under conditions which permits cleavage of at least a portion of said RNA molecules present in the sample thereby producing single repeats that form a hairpin structure based on their sense-antisens sequence information; contacting the formed hairpin structures with a second ToxN-A enzyme that cleaves the RNA in the loop structure to form double stranded RNA molecules; and
c. the resulting overhangs containing parts of the ToxN recognition sites is removed by using a standard single-strand specific ribonuclease such as RNase T1 thereby producing double stranded siRNAs. The method is illustrated in FIG. 12.

Preferably the ToxN endoribonucleases are the ToxN endoribonucleases as defined above.

Suitable RNA digestion buffers and reaction conditions for carry out cleavage of RNA is described in detail above.

### Method for Analyzing Modifications of RNA

The family of ToxN endoribonucleases are highly useful in RNA analysis in particular of long modified RNA molecules which have to be cleaved into fragments in order to be able to aid the analysis of the RNA modification.

Similar to the well-known DNA endoribonucleases enzymes commonly used in in vitro molecular cloning methods of double stranded DNA, the family of ToxN endoribonucleases is a group of enzymes that cleave single stranded RNA at specific recognition sequences without the need of any guide RNA, ribozymes or in complex with DNA probes.

5′capping of synthetically produced mRNA is crucial for an efficient in vivo translation of the mRNA into a functional protein or peptide.

However, capping of RNA in mRNA manufacturing is known to be an incomplete process leaving a portion of the produced mRNA uncapped. Completeness of capping can be assessed by methods such as LC-MS, IP RP HPLC, gel electrophoresis such as PAGE or capillary gel-electrophoresis (CGE).

The addition of a cap-structure changes the molecular weight of an RNA fragment at a level that is undetectable in standard molecular biology analytics. In addition, heterogeneity of an RNA product solution in terms of different product lengths makes it even more difficult to assess a change in molecular weight of the whole RNA population.

Thus, contemplated length harmonization of the whole RNA population would eventually yield only 2 to 5 sub-populations differing in terms of molecular weight dependent on their capping state.

Different types mRNA capping structures are well known to a skilled person and Chan, S. H. et al., RNase H-based analysis of synthetic mRNA 5′cap incorporation, RNA 2022, vol. 28, p.1144-1155 discloses several examples of 5′mRNA capping structures. Chan, S. H. et al. 2022 also discloses analysis of 5′capping efficiency of mRNA using DNA-RNA chimera-guided RNaseH cleavage of newly synthetically produced mRNA. However, a problem with RNaseH in this method is that it is difficult to get uniform cuts as the reaction has to be optimized both with regard to optimal DNA-RNA hybridization and the enzymes ability to provide a uniform cut. Another advantage with ToxN endoribonucleases is that they only require a pentamer as recognition site.

The use of ribozymes instead of RNase H has been suggested, EP3183340, however ribozymes requires excess amount of the enzyme and the enzyme is highly dependent on a 3D-structure of the RNA for cleavage.

Recently also capping of other RNA molecules has been detected in eukaryotes, bacteria and archaea. These noncanonical caps are primarily derived from metabolites and cofactors such as NAD⁺, FAD⁺ among others. The nonconical caps can affect RNA stability, mitochondrial function and RNA translation, Doamekpor et al. 2022 (https://www.ncbi.nlm.nih.gov/pmc/articles/PMC9283932/)

The inventors have shown for the first time that the family of ToxN endoribonucleases can overcome the problem of the endoribonucleases of the prior art, as the enzyme is able to digest single stranded RNA without the need of a hybridized DNA probe, a guide RNA or particular 3D structure of the RNA molecule for RNA cleavage.

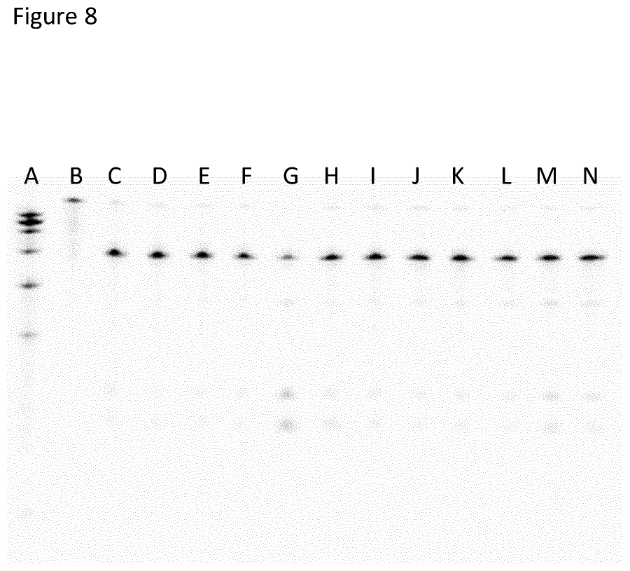

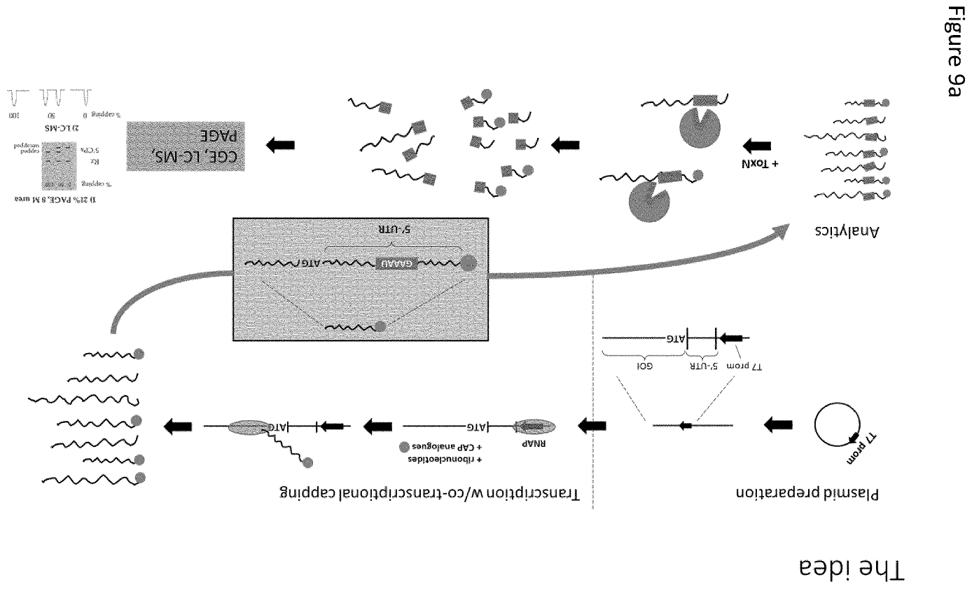

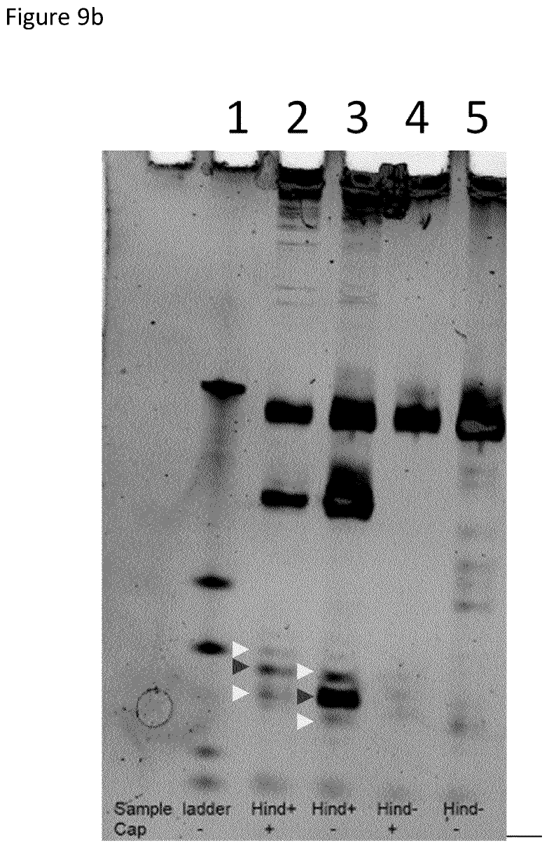

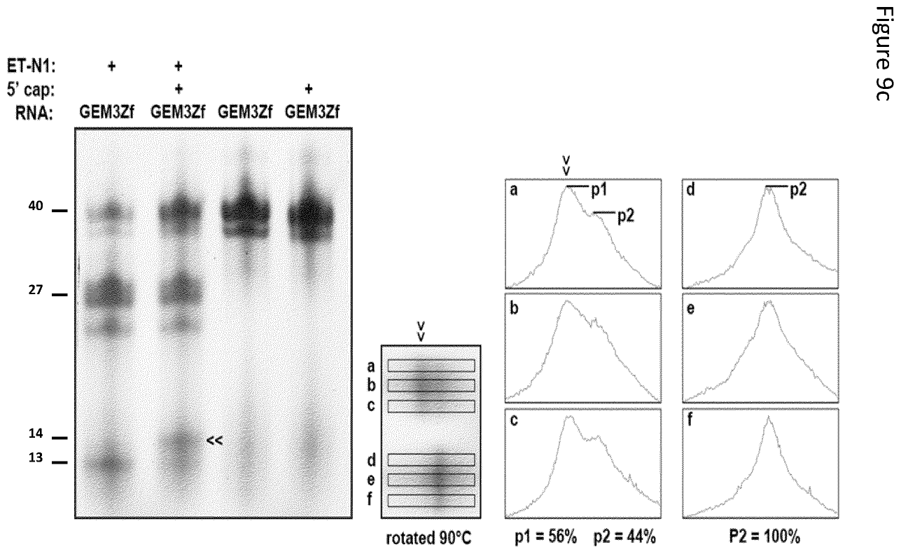

A method of analyzing the 5′capping efficiency of in vitro transcribed including co-transcriptional 5′capping of mRNA using ToxN enzyme is outlined in FIG. 9a. The mRNA is in vitro transcribed and includes a recognition site for a ToxN enzyme in the 5′UTR.

The ToxN recognition sequence is placed at a predefined number of ribonucleotides from the 5′end of the synthetically transcribed and 5′capped mRNA. Digestion of the 5′capped RNA with ToxN produced a population of 5′capped mRNA molecules of an equal ribonucleotide length.

For analysis of modifications of mRNA, the molecular weight difference provided by the modification is insignificant in long RNA molecules.

Long RNA molecules are RNA molecules of at least 10, 100, 200, 500 or 1000 nucleotides in length. Preferably the RNA molecule has a length from 5 to 50000 nucleotides, 10 to 30000 nucleotides, 100 to 25000 nucleotides or 200 to 20000 nucleotides, 500 to 15000 nucleotides.

In one aspect the 5′capped mRNA fragment generated after digestion with a ToxN enzyme comprises 2 to 100 ribonucleotides, preferably from 5 to 50 ribonucleotides and more preferably from 5 to 10 ribonucleotides.

In an alternative method a recognition sequence for a ToxN enzyme may be transcriptionally introduced on each side of a possible RNA fragment in question in order to investigate RNA modifications at internal positions of an RNA molecule.

In an alternative method a recognition sequence for a ToxN enzyme may be transcriptionally introduced in front of the Poly(A)-tail of a possible mRNA fragment in question in order to investigate the Poly(A)-tail length distribution of an mRNA molecule.

Thus, in one aspect there is provided a method of determining 5′capping efficiency of RNA molecule modification wherein the method comprises the steps:
a. providing a sample comprising at least one single stranded mRNA molecule comprising a cleavage site for a ToxN endoribonuclease in the 5′UTR of said mRNA;
b. contacting said sample from step a) with a ToxN endoribonuclease or an enzymatic active fragment thereof under conditions that permits cleavage of at least a portion of said single stranded RNA molecules to produce at least one 5′ terminal RNA fragment and at least one 3′ terminal RNA fragment.
c. separating the RNA fragments from step b) and determine the presence of a 5′-capping modification at the 5′end of said 5′ terminal RNA fragment.

In a further aspect the separation and detection of step c) of the above method is based on different molecular weight, charge or length of the generated RNA fragments.

Preferably the ToxN endoribonucleases are the ToxN endoribonucleases as defined above.

Suitable RNA digestion buffers and reaction conditions for carry out cleavage of RNA is described in detail above.

Thus, in another aspect there is provided a method of determining Poly(A)-tail length distribution of RNA molecules wherein the method comprises the steps:
a. providing a sample comprising at least one single stranded mRNA molecule comprising a cleavage site for a ToxN endoribonuclease in the 3′UTR of said mRNA located upstream of the Poly(A)-tail;
b. contacting said sample from step a) with a ToxN endoribonuclease or an enzymatically active fragment thereof under conditions that permits cleavage of at least a portion of said single stranded RNA molecules to produce at least one 5′ terminal RNA fragment and at least one 3′ terminal RNA fragment.
c. separating the RNA fragments from step b) and determine the presence and length of a Poly(A) tail at the 3′end of said 3′ terminal RNA fragment.

The 3′ terminal RNA fragments comprising poly(A)-tails may be enriched by using an oligo-dT based enrichment step thereby removing the non-Poly(A) containing fraction.

Preferably the ToxN endoribonucleases are the ToxN endoribonucleases as defined above.

Suitable RNA digestion buffers and reaction conditions for carry out cleavage of RNA is described in detail above.

In a further aspect the separation and detection of step c) of the above method is based on different molecular weight, charge or length of the generated RNA fragments.

In a further aspect separation and detection of the RNA fragments is selected from gel electrophoresis, capillary electrophoresis, high pressure liquid chromatography (HPLC), mass spectrometry (MS) or LC-MS, LC-UV.

As described above capping analysis after cleavage with E. coli ToxN1 can be performed the same way as the other methods with LC-MS, FFF (Fastflow Fractionation) or similar techniques. The pentamer recognition site is quite versatile and can be placed anywhere in the 5′UTR. The enzyme does not require an overhang and cuts ssRNA that starts with the cleavage sites. The shortest fragment that EcoToxN1 can produce is therefore three nucleotides long: GAA when cutting directly at the 5′ site, see Table 1 above.

Since no additional oligoes in excess are present in capping analysis with E. coli ToxN1 the analysis can be done on a simple urea/acrylamide gel. Example 10a-c and FIG. 9b-d shows that the ToxN endoribonuclease may be used in a method for analysing RNA modifications such as for example 5′ capping of RNA.

### Method for RNA Fingerprinting

Similar to DNA fingerprinting with DNA restriction enzymes, the RNA restriction enzymes may be used to exploit the different location of cleavage sites in a ssRNA. RNA fingerprinting is an important tool within diagnostics and therapeutics. ToxN endoribonucleases for use in analytically RNA fingerprinting has several advantages including a fast and reliable fragmentation of single stranded RNA molecules of defined length that can be analysed by gel electrophoresis, capillary electrophoresis, high pressure liquid chromatography (HPLC), mass spectrometry (MS) or LC-MS, LC-UV.

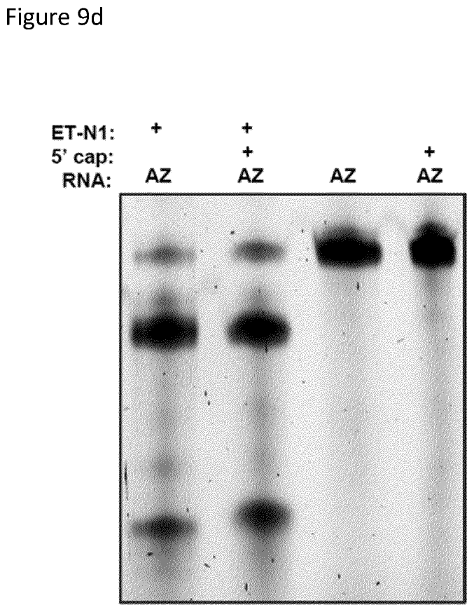

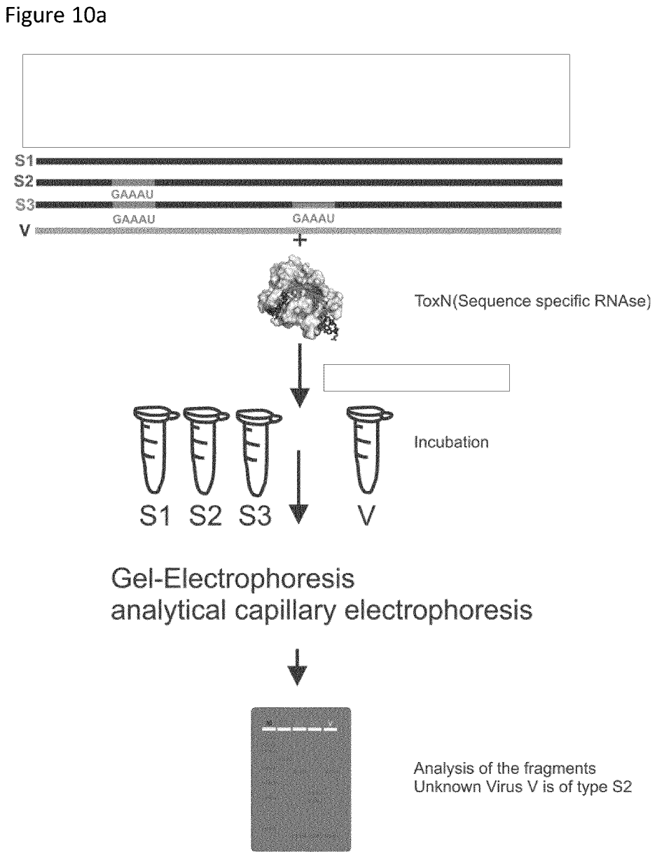

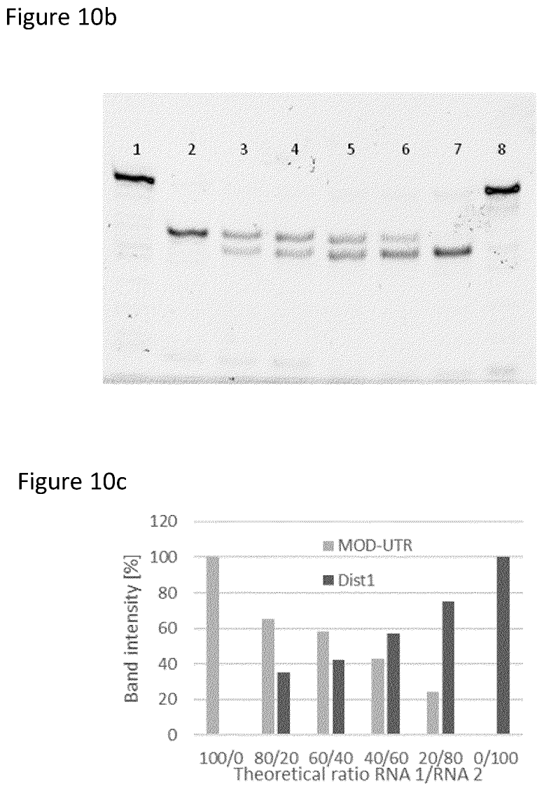

There is provided a method of RNA fingerprinting, illustrated in FIG. 10a, wherein the method comprises the steps:
a. providing a sample comprising a single stranded RNA molecule with unknown sequence;
b. contacting said sample from step a) with a ToxN endoribonuclease or an enzymatic active fragment thereof under conditions that permit cleavage of at least a portion of the RNA molecules present in said sample obtaining a plurality of RNA fragments;
c. separation and detection of the fragmented RNA molecules from step b) thereby obtaining a fingerprint of said RNA molecule with unknown sequence and compare the obtained fingerprint with fingerprints from RNA molecules with known RNA sequence.

Preferably the ToxN endoribonucleases are the ToxN endoribonucleases as defined above.

Suitable RNA digestion buffers and reaction conditions for carry out cleavage of RNA is described in detail above.

Determination of RNA species within a multivalent RNA composition as described in WO2022212711, is another RNA fingerprinting method wherein the use of a ToxN endoribonuclease will simplify and improve the method over RNase H enzymes that require a hybridized DNA probes at their recognition site.

The method is based on RNA compositions comprising one or more distinct RNA species (e.g, RNAs encoding different proteins), where each RNA species comprises a unique nucleotide sequence that can be used to identify the RNA species. The incorporation of unique identification and/or ratio determination (IDR) sequences into distinct RNA species of a RNA composition in at least one position inferring a unique fingerprint to each RNA species (e.g., within a non-coding region).

From WO2022212711 and without wishing to be bound by theory, it is believed that RNAs can be digested to release RNA fragments comprising the IDR sequence, and analytical methods can be used to quantify the types and amounts of RNA fragments containing each IDR, to generate a profile of the types and/or amounts of each RNA species in a RNA composition.

Use of IDR sequences for analysis allows characterization of multivalent RNA compositions comprising several distinct RNA species, even if multiple RNA species are difficult to distinguish by length or coding sequence. For example, a multivalent RNA composition comprising eight RNA species, each encoding a different serotype of the same protein, may have similar lengths and coding sequences, but each RNA species may comprise a different IDR pattern in a coding or non-coding region.

Because each IDR sequence unambiguously identifies a particular RNA species, the abundance of IDR sequences may be measured to determine the abundance of RNAs encoding each serotype.

Furthermore, the coding sequence of one or more RNA species in a multivalent RNA composition may be modified (e.g., to alter the structure of an encoded therapeutic protein or antigen) independently from the IDR sequence, such that the same analytical methods may be used to evaluate a RNA composition in which one or more RNA coding sequences are modified.

Thus, there is provided a method for analysing a RNA species in a multivalent RNA composition, wherein the method comprises the steps:
a. providing a sample comprising a multivalent RNA composition comprising a first RNA species and a second RNA species comprising a cleavage site for a ToxN endoribonuclease in at least one position of said first RNA species and a second RNA species;
b. contacting said sample from step a) with a ToxN endoribonuclease under conditions that permit cleavage of said first RNA species and a second RNA species thereby releasing a plurality of first RNA fragments and second RNA fragments from the first and second RNA species;
c. separation and detection the presence and/or amount of the released first RNA fragments and second RNA fragments.

The RNA molecule may be selected from mRNA, viral RNA, invitro transcribed mRNA, therapeutic RNA.

Preferably the ToxN endoribonucleases are the ToxN endoribonucleases as defined above.

Suitable RNA digestion buffers and reaction conditions for carry out cleavage of RNA is described in detail above.

The separation and detection of step c) of the above method may be based on different molecular weight, charge or length of the generated RNA fragments.

In a further aspect separation and detection of the RNA fragments is selected from gel electrophoresis, capillary electrophoresis, high pressure liquid chromatography (HPLC), mass spectrometry (MS) or LC-MS, LC-UV.

Example 11 and FIGS. 10b and 10c shows that endoribonucleases from Type III toxin-antitoxin (TA) system such as endoribonucleases from the ToxN subfamily may be used in a method for RNA fingerprinting. FIG. 10b depicts two synthetic RNA oligoes of identical length of 20 bases that migrate equally in the urea/acrylamide gel (lanes 1 and 8). However, each RNA oligo has the E. coli ToxN1 (ET-N1) cleavage site located at different position in the RNA sequence. Thereby, the RNA molecules present in the mixture can be identified by their unique pattern of RNA fragments. Furthermore, the band intensity can be used to estimate the ratio of the two RNA molecules in the mixture, cf. FIG. 10c.

Having generally described this invention, a further understanding can be obtained by reference to certain specific examples. The examples illustrate the properties and effects of the of the ToxN endoribonucleases according to the invention, and are provided herein for purposes of illustration only, and are not intended to be limiting.

### EXAMPLES

### Example 1—Cloning, Expression and Purification of E. coli ToxN1 (ET-N1) Endoribonuclease with SEQ ID NO: 1

The E. coli ToxN1 (ET-N1) enzyme (toxin) was cloned into pBAD/His A vector and the RNA (antitoxin) was cloned into pRSFDuet™-1 as described in Manikandan, P. et al., Identification, functional characterization, assembly and structure of ToxIN type III toxin-antitoxin complex from E. coli, Nucleic acid research, 2022, vol. 50, no.3, p.1687-1700. The plasmids containing toxin and antitoxin were co-transformed into E. coli BL21(DE3) cells and grown overnight at 37° C., 180 rpm, followed by the secondary culture at 37° C., 180 rpm till OD600~0.5. The culture was incubated at 15° C. without shaking for 30 min and the toxin was induced by adding IPTG to a final concentration of 1 mM and incubated at 15° C., 180 rpm for 24 h. The cells were harvested by centrifugation at 6000 rpm for 15 min. The cells were resuspended in lysis buffer (50 mM Tris, 300 mM NaCl, 10 mM imidazole, 10% glycerol, 2 mM 2-mercaptoethanol pH 7.5 at 25° C.) and lysed by sonication. The lysate was centrifuged at 13 000 rpm for 30 min, and the supernatant was loaded on a Ni²⁺-NTA column. The complex was eluted using elution buffer (lysis buffer+200 mM imidazole). Fractions containing the complex were dialyzed against ion-exchange buffer (50 mM NaCl, 50 mM Tris-HCl, 1 mM DTT pH 7.5) and purified using anion exchange chromatography by increasing gradient of NaCl from 50 to 1000 mM, over a volume of 100 ml, which yielded separate fractions of toxin, −E. coli ToxN1 (ET-N1) (at ~300 mM NaCl), antitoxin, RNA (at ~600 mM NaCl) and complex of E. coli ToxN1-RNA (at ~500 mM NaCl). They were further purified by size exclusion chromatography (SEC) using an S200 column (GE).

Toxin (E. coli ToxN1)_Fwd
5′-AGGTCATATGGCGAAATTTTTCACAATATCA-3′
Toxin (E. coli ToxN1)_Rev
5′-GGAAATAGACGATCTCGAGTCTAGAGCAT-3′

### Example 2—In Vitro Transcription and Production of RNA Oligoes Comprising a ToxN Recognition and Cleavage Site

In vitro transcription was done in 50 μL reactions with 30U T7 RNA Polymerase (ThermoScientific, EPO111) using the included 5× reaction buffer (200 mM Tris-HCl pH 7.9; 30 mM MgCl2, 50 mM DTT, 50 mM NaCl, 10 mM spermidine), 50U RiboLock RNase Inhibitor (ThermoScientific, E00381), 2 mM NTPs (ThermoScientific, R1481), and 1 μg linearized (ScaI digested) pGEMEX-1 template. As alternative templates any linearized plasmids with a T7 promoter and ToxN cleavage sites or PCR products including a T7 promoter and ToxN cleavage sites can be used. The transcription reaction lasted for 2 h at 37° C. and was inactivated by adding 10 μL 60 mM ETDA followed by a 10 min incubation at 65° C. Reaction cleanup was conducted using the RNeasy MiniElute Cleanup Kit (Qiagen, 74204) to elute the purified RNA in RNase free water.

### Recognition Sites:

E. coli ToxN (ET-N1): GAA\hat{ }AU
P. atrosepticum ToxN: GAA\hat{ }AU
B. Thuringiensis ToxN (BT-N1): AAA\hat{ }AAA
L. lactis ToxN (AbiQ): AA\hat{ }AA
[Eubacterium] rectale CptIN: GA\hat{ }AAG
Escherichia albertii ToxN/AbiQ (ET-N5): 5′ . . . GAAAJ⬇AAC . . . 3′ and 5′. AAAA⬇AUC . . . 3′

### Example 3—Endoribonuclease Activity—Determining Optimal Enzyme Concentration

The experiment was performed in order to verify that the E. coli ToxN1 (NCBI Acc. No.: WP_059274511) of use according to the invention exhibit endoribonuclease activity on a single-stranded RNA substrate.

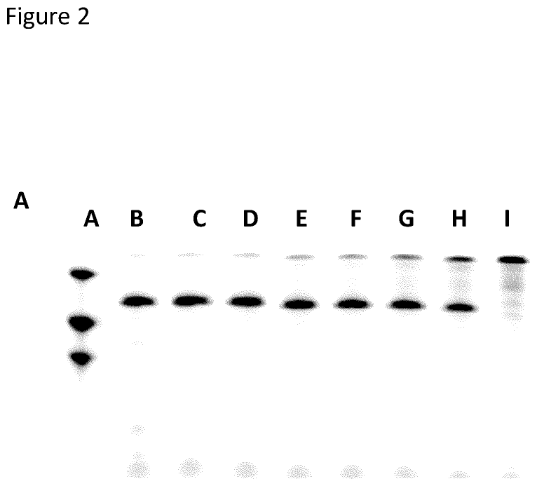

Digestion of a 50 nucleotide RNA single strand oligo with E. coli ToxN1. The recognition site GAAAU is in the centre of the oligo and results in a product of 26 nucleotides. Results from analysis on a Polyacrylamid/Urea gel are shown in FIG. 2.

Sequence of the 50 nucleotide RNA single strand oligo with cleavage site for E. coli ToxN1 (ET-N1) underlined:

5′-CGCAAUUGCCGCAUUACAAGGGAAAUAACUUCGUACGUUGUUGUUAGCAU/3′-FAM

### Assay Conditions (25 μl)

0 nM to 120 nM ToxN enzyme
50 mM Tris-HCl pH 7.5
50 mM NaCl
1 mM DTT
0.125 μM RNA oligo
1 h at 15° C.

### Example 4—Activity Profiling of E. coli ToxN1 (ET-N1) Endoribonucleases: Concentration of Monovalent Salt and pH

Digestion of a 50 nucleotide RNA single strand oligo with ToxN. The recognition site GAAAU is in the centre of the oligo and results in a product of 26 nucleotides (the sequence of the oligo is shown in Example 3). The results are shown in FIG. 3 and demonstrates that the ToxN enzyme are able to cleave single stranded RNA at pH ranging from 7.0 to 9.0. The results further demonstrate that the specificity of the enzyme is dependent on monovalent ion concentration and is optimal at NaCl concentrations of 50 mM and lower. Above 50 mM the cleavage of the RNA is less specific and the enzyme digests RNA at secondary closely related sequence recognition sites.

### Assay Conditions

125 nM RNA oligo
40 nM E. coli ToxN1
50 mM Tris-HCl pH 7.0 to pH 9.0
NaCl 0 mM to 175 mM
1 h at 15° C.
   Results from Analysis on a Polyacrylamide/Urea Gel

### Example 5—Activity Profiling of ToxN Endoribonucleases: Concentration of Divalent Salt

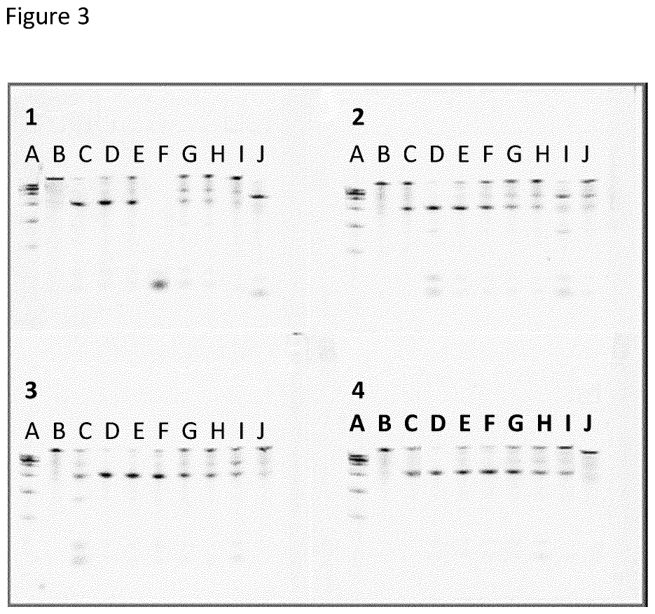

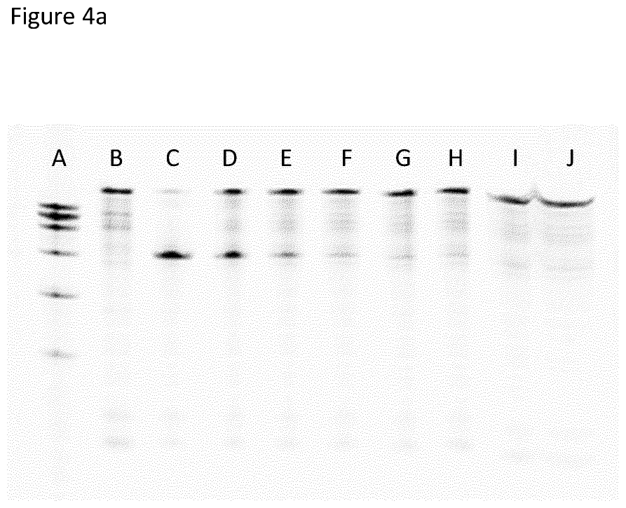

a) Digestion of a 50 nucleotide RNA single strand oligo with E. coli ToxN1 (ET-N1). The recognition site GAAAU is in the centre of the oligo and results in a product of 26 nucleotides (the sequence of the oligo is shown in Example 3). The results are shown in FIG. 4a. E. coli ToxN1 (ET-N1) enzyme activity is extensively inhibited by concentrations of MgCl₂ above 1 mM (lane E-lane J).

| A | B | C | D | E | F | G | H | I | J |
|---|---|---|---|---|---|---|---|---|---|
| MW | w/o | ToxN | ToxN | ToxN | ToxN | ToxN | ToxN | ToxN | ToxN |
| DNA | ToxN | 0 mM | 1 mM | 2 mM | 3 mM | 4 mM | 5 mM | 6 mM | 7 mM |
| ladder | enzyme | MgCl₂ | MgCl₂ | MgCl₂ | MgCl₂ | MgCl₂ | MgCl₂ | MgCl₂ | MgCl₂ |

### Assay Conditions

50 mM TrisHCl pH 8.0
25 mM NaCl
125 nM RNA oligo
40 nM E. coli ToxN1
1 h at 15° C.

b) Digestion of a 50 nucleotide RNA single strand oligo with ToxN. The recognition site GAAAU is in the centre of the oligo and results in a product of 26 nucleotides (the sequence of the oligo is shown in Example 3). The results are shown in FIG. 4b. ToxN enzyme activity is extensively inhibited by concentrations of MgCl₂ above 3 mM and of MnCl₂ above 1 mM.

### Assay Conditions

25 mM Tris/HCl pH 7.5
25 mM NaCl
0.5 μM RNA oligo
10 nM E. coli ToxN1
10 min at 37° C.

### Example 6—Activity Profiling of E. coli ToxN1 (ET-N1) Endoribonucleases: Concentration of Divalent Salt, EDTA and DTT

Digestion of a 50 nucleotide RNA single strand oligo with ToxN1 recognition sequence. The recognition site GAAAU is in the centre of the oligo and results in a product of 26 nucleotides (the sequence of the oligo is shown in Example 3).

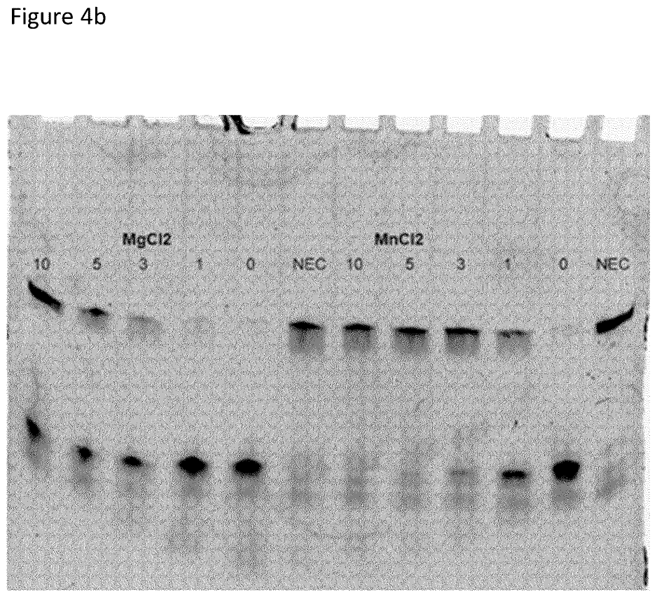

The results from the experiment is shown in FIG. 5 which demonstrates that ToxN1 endoribonuclease activity is regained by the addition of EDTA, a divalent cation scavenger (lanes G, H).

| A | B | C | D | E | F | G | H | | I | J | |
|---|---|---|---|---|---|---|---|---|---|---|---|
| MW | w/o | ToxN | ToxN | ToxN | ToxN | ToxN | ToxN | | ToxN | ToxN | |
| DNA | ToxN | | 5 mM | 5 mM | 5 mM | 5 mM | 5 | mM | 5 mM | 5 | mM |

| | | | | | | | | |
|---|---|---|---|---|---|---|---|---|
| ladder | enzyme | MgCl₂ | EDTA | DTT | MgCl₂ | MgCl₂ | MgCl₂ | MgCl₂ |

| | | | | | |
|---|---|---|---|---|---|
| 5 mM | 10 | mM | 5 mM | 10 | mM |

| | | | |
|---|---|---|---|
| EDTA | EDTA | EDTA | EDTA |

| | | |
|---|---|---|
| 5 mM | 5 | mM |

| | |
|---|---|
| DTT | DTT |

### Assay Conditions

50 mM TrisHCl pH 8.0
25 mM NaCl
125 nM RNA oligo
40 nM E. coli ToxN1 (ET-N1)
1 h at 15° C.

### Example 7—Activity Profiling of E. coli ToxN1 (ET-N1) Endoribonucleases: Incubation Time at 15° C.

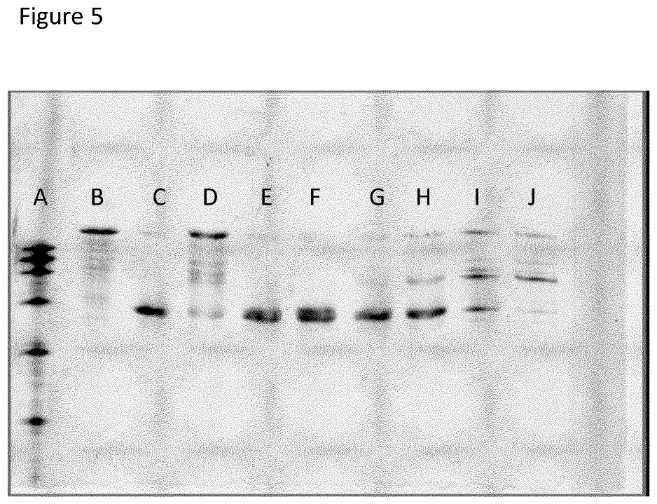

Digestion of a 50 nucleotide RNA single strand oligo with E. coli ToxN1 (ET-N1) at increasing incubation time. The recognition site GAAAU is in the centre of the oligo and results in a product of 26 nucleotides (the sequence of the oligo is shown in Example 3). The results are shown in FIG. 6 and demonstrates that the enzyme is efficient. 50% of the RNA oligos are cleaved after 5 min at an optimal assay temperature of 15° C. (lane C).

| A | B | C | D | E | F | G | H | I | J |
|---|---|---|---|---|---|---|---|---|---|
| MW | w/o | ToxN1 | ToxN1 | ToxN1 | ToxN1 | ToxN1 | ToxN1 | ToxN1 | ToxN1 |
| DNA | ToxN1 | 5 min. | 10 min. | 15 min. | 20 min. | 30 min. | 40 min. | 50 min. | 60 min. |
| ladder | enzyme | | | | | | | | |

### Assay Conditions

50 mM Tris-HCl pH 8.0
25 mM NaCl
125 nM RNA oligo
40 nM ToxN1 (ET-N1)
15° C.

### Example 8—Activity Profiling of ToxN Endoribonucleases: Incubation Temperature

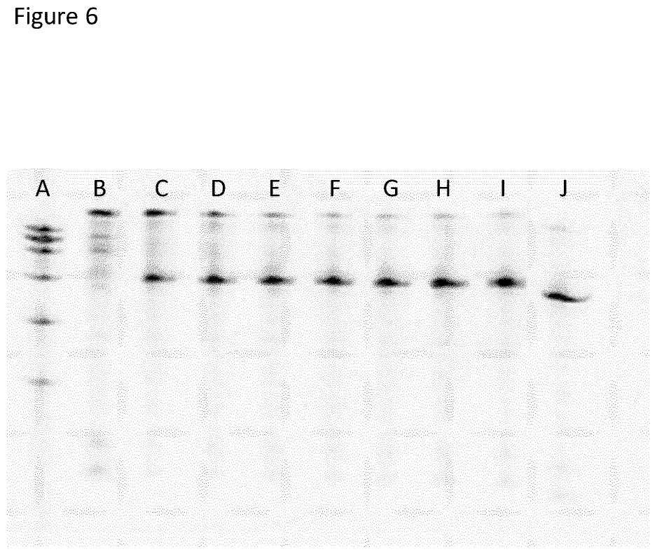

Digestion of a 50 nucleotide RNA single strand oligo with E. coli ToxN1 (ET-N1). The recognition site GAAAU is in the centre of the oligo and results in a product of 26 nucleotides (the sequence of the oligo is shown in Example 3). The aim of the study was to profile the enzyme activity at different incubation temperatures. The results are shown in FIG. 7.

| A | B | C | D | E | F | G | H | I |
|---|---|---|---|---|---|---|---|---|
| MW | w/o | ToxN1 | ToxN1 | ToxN1 | ToxN1 | ToxN1 | ToxN1 | ToxN1 |
| DNA | ToxN1 | 6° C. | 7° C. | 10° C. | 13° C. | 17° C. | 22° C. | 27° C. |
| ladder | enzyme | | | | | | | |

50 mM TrisHCl pH 8.0
25 mM NaCl
125 nM RNA oligo
40 nM ToxN1 (ET-N1)
30 min at the specified temperatures

### Example 9—Activity Profiling of E. coli ToxN1 Endoribonucleases: Incubation Temperature

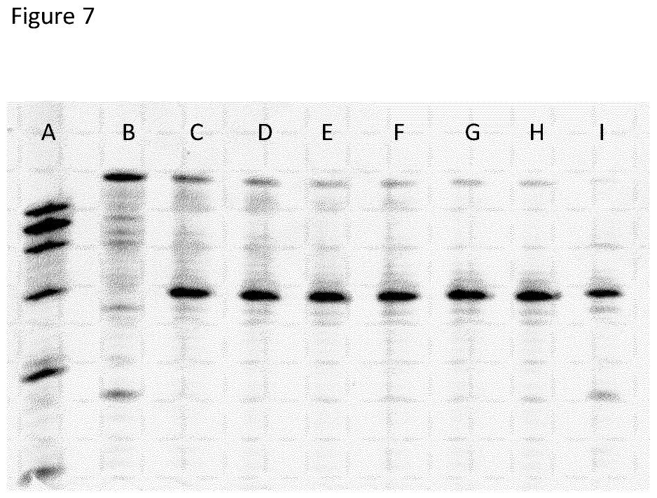

Digestion of a 50 nucleotide RNA single strand oligo with E. coli ToxN1 (ET-N1). The recognition site GAAAU is in the centre of the oligo and results in a product of 26 nucleotides (the sequence of the oligo is shown in Example 3). The aim of the study was to profile the enzyme activity at different incubation temperatures. The results are shown in FIG. 8 and demonstrates that the enzyme specificity decreases at temperatures above 30° C.

| A | B | C | D | E | F | G | H | I |
|---|---|---|---|---|---|---|---|---|
| MW | w/o | ToxN1 | ToxN1 | ToxN1 | ToxN1 | ToxN1 | ToxN1 | ToxN1 |
| DNA | ToxN | 15° C. | 16° C. | 17° C. | 20° C. | 23° C. | 27° C. | 32° C. |
| ladder | enzyme | | | | | | | |

| J | K | L | M | N |
|---|---|---|---|---|
| ToxN1 | ToxN1 | ToxN1 | ToxN1 | ToxN1 |
| 37° C. | 40° C. | 43° C. | 44° C. | 45° C. |

### Assay Conditions

50 mM TrisHCl pH 8.0
25 mM NaCl
125 nM RNA oligo
40 nM ToxN1 (ET-N1)
30 min at the specified temperatures

### Example 10a—Analysis of 5′-Cap Status

In vitro translation (IVT) for the production of an RNA construct was performed using standard protocol protocols.

Digestion of the IVT RNA was performed at 37° C. After stopping the IVT reaction with EDTA, a fraction of the IVT reaction was mixed with 10-90 nM E. coli ToxN1 (ET-N1) for 10-20 min in IVT buffer. The reaction was stopped by adding urea gel loading buffer (95% Formamide, 0.25 M EDTA, Bromophenol blue) and the samples loaded on a 20% urea/acrylamide gel. The samples were visualized with SYBRGold.

The results from the analysis of 5′-cap status is depicted in FIG. 9b.

### Example 10b—Analysis of Post-Transcriptional Capping Efficiency of mRNA

pGEM3Zf-ETN1+13 was linearized with HincII and used in a T7 RNAP IVT reaction. Half of the resulting GEM3Zf mRNA was then capped using a vaccinia capping enzyme and purified on a silica column. Next, half of the capped and uncapped mRNA was digested using the E. coli ToxN1 (ET-N1) endoribonuclease and loaded on a 10% polyacrylamide gel containing 7M urea (TBE, Sybr Gold). FIJI was used to quantify the relative band intensity of capped (<<p1) and uncapped (p2) mRNA fragments (averaged peak heights of three slices, indicated as “a, b, c” for capped mRNA and “d, e, f” for uncapped mRNA). About 150 ng mRNA was loaded per lane after 1:1 dilution in RNA loading buffer and heating 10 minutes at 65° C.

### Construct Used in IVT:

pGEM3Zf:
TAATACGACTCACTATA_GGGCGAATTCGAA↓ATCGGTACCCGGGGATCCT
CTAGAGTC*GAC
Resulting in mRNA GEM3Zf:
(5′cap)GGGCGAAUUCGAA↓AUCGGUACCCGGGGAUCCUCUAGAGUC

⬇ToxN/E. coli ToxN1/(ET-N1) cleavage site
*HincII cleavage site (for plasmid linearization)
_indicates transcription start

The results from the analysis of 5′-cap status is depicted in FIG. 9c. This method indicates that 56% of the mRNA is capped.

### Example 10c—Analysis of Co-Transcriptional Capping Efficiency of mRNA

pAZ_01 was linearized with PpuMI and used in a T7 RNAP IVT reaction with and without co-transcriptional capping with cap analogs. Half of the capped and uncapped pAZ_01 mRNA was digested using the E. coli ToxN1 (ET-N1) endoribonuclease and loaded on a 10% polyacrylamide gel containing 7M urea (stained with Sybr Gold). Uncapped mRNA and capped mRNA that was digested with ET-N1 shows one band of larger size thereby suggesting near to 100% capping efficiency. About 150 ng mRNA or 15 ng RNA oligos were loaded per lane after 1:1 dilution in RNA loading buffer and heating 10 minutes at 65° C.

### Construct Used in IVT:

pAZ_01:
TAATACGACTCACTATA_AGGTCTTCTGGTCCCCACAGAA↓ATCTCAGAGA
GAACCCACCATGGAGGACGCAAAGAACATAAAAAAAG*GACCC
Resulting in mRNA AZ_01:
(5′cap)AGGUCUUCUGGUCCCCACAGAA↓AUCUCAGAGAGAACCCACCAU
GGAGGACGCAAAGAACAUAAAAAAAG

⬇ ToxN/E. coli ToxN1/(ET-N1) cleavage site
*PpuMI cleavage site (for plasmid linearization)
_indicates transcription start

The results from the analysis of 5′-cap status is depicted in FIG. 9d.

### Example 11—Fingerprinting by Analysis of Ratios of RNA Mixtures

RNA Fingerprinting by digestion of two 20 nucleotide RNA substrates simultaneously (MOD-UTR: GGGAAJAUAAGAGAGAAAAGA-FAM and Dist1: GCCGAAJAUAGUGACCCUGCA-FAM). The FAM labelled products differ by just one nucleotide resulting in product band of 15 and 14 nucleotides respectively. Reaction conditions: 10 min at 37° C. with 10 nM E. coli ToxN1 (ET-N1), 500 nM substrate. Only the product with the FAM label is visible.

The results from the fingerprint analysis is depicted in FIG. 10b. FIG. 10c depicts the ratio of the two RNA molecules in the mixture calculated by measuring the band intensities.

### Example 12—RNA Synthesis

Mango aptamer production. An IVT produced concatemer of the Mango aptamer was digested with E. coli ToxN1 (ET-N1), at decreasing enzyme concentrations in a buffer comprising 25 mM Tris/HCl, 25 mM NaCl pH 7.5, for 20 min at 37 degrees Celsius. The Mango aptamer is described in Dolgosheina et al., 2014; doi: 10.1021/cb500499x.

### Example 13—Stability of the E. coli ToxN1 (ET-N1) Enzyme in the Presence of Monovalent Salt in the Storage Buffer 100 mm NaCl to 500 mM NaCl

### Example 14—Digestion of RNA by Other ToxN Endoribonucleases of the ToxIN Family

25 mM Tris pH 7.5, 25 mM NaCl
10 min at 37 degrees Celcius
500 nM RNA substrate
10 nM endoribonuclease
20 μL total volume

### Substrate Oligos:

MOD-UTR
GGGAA↓AUAAGAGAGAAAAGA-FAM
RS2
AGACAGAUCGAAA↓AACAUGU-FAM
RS3
AGACAUAUCAAA↓AAAUCGUA-FAM
Q1
6-FAM-AGACAGAA↓AUGCAU/36-TAMSp

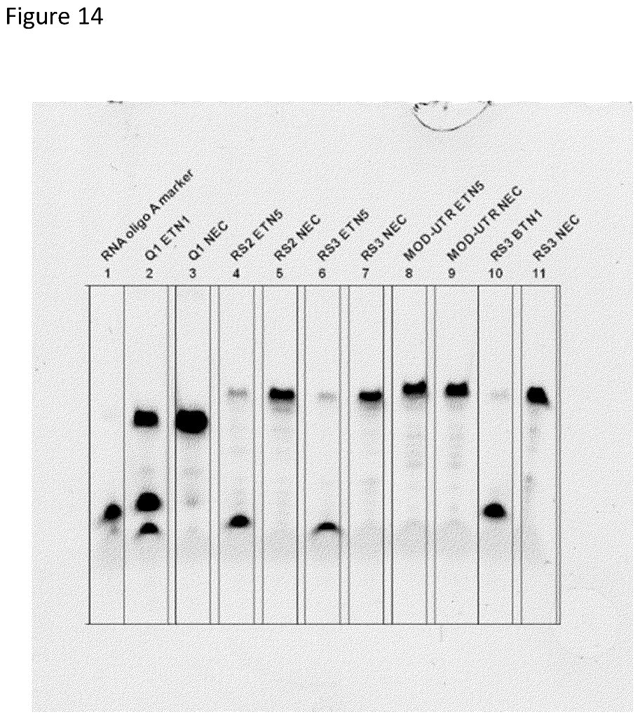

The results are shown in FIG. 14. BT-N1 recognizes and cleaves with good confidence AAAJAAA. ET-N5 cleaves GAAA⬇AAC and AAAA⬇AUC with similar efficiency.

This example demonstrates that further members of endoribonucleases belonging to the family of ToxN enzymes according to pFam classification also cuts single stranded RNA under the same buffer conditions as ToxN (ET-N1) even though the protein sequence % identity between ET-N1 and ET-N5 is only 40.88 and protein sequence % identity between ET-N1 and BT-N1 is only 30.95%. The % identity is calculated using Clustal Omega, default settings, multiple protein sequence alignment tool from EMBL-EBI.

| % | | | |
|---|---|---|---|
| identity | BT-N1 | ET-N1 | ET-N5 |
| BT-N1 | 100 | 30.95 | 30.97 |
| ET-N1 | 30.95 | 100 | 40.88 |
| ET-N5 | 30.97 | 40.88 | 100 |

### SEQUENCE LISTING

| SEQ ID | | |
|---|---|---|
| NO | NCBI or UniProt Acc. No. | Description |
| 1 | PDB: 7D8O_A | Amino acid |
| | MAKFFTISSSYIKYLKDFDDKVPNSEDPTYNNPK | sequence type |
| | AFIGIVLEIEGHKYLAPLTSPKAWHANVKESSPA | III toxin- |
| | FFKLHENGVPDNQLGLINLKFMIPIIEAEVSLLD | antitoxin |
| | LDSMPDTPYKRMLYKQLQFIRVNEDKISEKSKLL | system |
| | RNLALQGRMQGTCDFAVLEEKYQHFGKKPEDM | ToxN/AbiQ |
| | EIDDLESR | family |
| | | toxin |
| | | [Escherichia] |
| 2 | PDB: 7D8O_A | Amino acid |
| | MNHKVHHHHHHMAKFFTISSSYIKYLKDFDDKV | sequence type |
| | PNSEDPTYNNPKAFIGIVLEIEGHKYLAPLTSP | III toxin- |
| | KAWHANVKESSPAFFKLHENGVPDNQLGLINLK | antitoxin |
| | FMIPIIEAEVSLLDLDSMPDTPYKRMLYKQLQF | system |
| | IRVNEDKISEKSKLLRNLALQGRMQGTCDFA | ToxN/AbiQ |
| | VLEEKYQHFGKKPEDMEIDDLESR | family |
| | | toxin |
| | | [Escherichia] |
| | | incl. N-terminal |
| | | His-tag |
| 3 | ATGAAATTTTTCACAATATCAAGCAGTTACATA | DNA sequence |
| | AAATACCTGAAGGACTTTGATGACAAAGTTCCCA | encoding E.coli |
| | ATAGCGAAGATCCTACATACAACAATCCTAAGGCT | ToxN1 with |
| | TTCATTGGCATAGTACTAGAGATTGAAGGACATA | amino acid |
| | AATATTTAGCACCTTTAACATCGCCAAAAGCATGG | sequence with |
| | CATGCTAATGTAAAAGAGTCATCACCCGCCTTCTT | SEQ ID No. 1 |
| | CAAACTTCATGAAAACGGTGTCCCTGATAATCAGC | and DNA |
| | TTGGATTGATTAATCTGAAATTTATGATTCCAATAA | sequence |
| | TCGAAGCTGAAGTGTCTTTACTTGATCTGGATAGC | |
| | ATGCCTGATACGCCTTACAAAAGAATGCTATATAA | |
| | ACAGTTACAATTTATTCGTGTAAATGAAGATAAAA | |
| | TATCTGAAAAATCAAAACTATTAAGAAACCTTGCC | |
| | TTGCAAGGCAGAATGCAAGGAACATGCGACTTTGCC | |
| | GTGTTAGAAGAAAAATATCAACATTTCGGTAAAAAA | |
| | CCTGAAGACATGGAAATAGACGATTAA | |
| 4 | AGGTGATTTGCTACCTTTAAGTGCAGCTAGAA | DNA sequence |
| | ATTTAGGTGATTTGCTACCTTTAAGTGCAGCTAG | ToxI antitoxin |
| | AAATTTAGGTGATTTGCTACCTTTAAGTGCAGCTA | repeats |
| | GAAATTCAGGTGATTTGCTACCTTTAAGTGCAGCT | |
| | AGAAATTTAGGTCATTTACTACCTTAAAGT | |
| 5 | AUUCAGGUGAUUUGCUACCUUUAAGUGCAGCUAGAA | RNA molecule |
| | | ToxI antitoxin |
| | | single strand |
| | | repeat of ToxIN |
| | | complex with |
| | | toxin ET-N1 |
| 6 | B8X8Z0 | Amino acid |
| | MKFYTISSKYIEYLKEFDDKVPNSEDPTYQNPKA | sequence type |
| | FIGIVLEIQGHKYLAPLTSPKKWHNNVKESSLSC | III toxin- |
| | FKLHENGVPENQLGLINLKEMIPIIEAEVSLLDL | antitoxin |
| | GNMPNTPYKRMLYKQLQFIRANSDKIASKSDTLRN | system |
| | LVLQGKMQGTCNFSLLEEKYRDFGKEAEDTEEGE | ToxN/AbiQ |
| | | family toxin |
| | | [Pectobacterium |
| | | atrosepticum] |
| 7 | Q3YN09 | Amino acid |
| | MTNKDNPKFHTISTEYIDYLREADSKVPENKDEQHSRP | sequence type |
| | YVGVLEKINGHDYFVPLTSRNDKNFNSQVSVKLFDND | III toxin- |
| | EKRIGVLLVNNMIPVPEKECKEIDIAEKTAADPQYGN | antitoxin |
| | LMLKQYLFLKENMDRVTNKVEKVYKDVTVQGKPSHKQ | system |
| | KFLKGVCCDFPKLEEKCQEYKERDQAKERDKARRIAY | ToxN/AbiQ |
| | MRQMGRER | family toxin |
| | | [Bacillus cereus |
| | | group] (BT-N1) |
| 8 | Q9ZJ19.1 | Amino acid |
| | MSSFFYKEILRMTLRFFTVTDEYIAYLRKFESKV | sequence type |
| | HYQYENNASTYVGVVLKKNDFNYFIPLSSYKKGN | III toxin- |
| | PEKDKAMKKRSRIVTRLFEIGNINNPLGYLLHHNM | antitoxin |
| | IPVPDSELIPLPLDLKKPKHKMMQKQLIYMKSISE | system |
| | KIENKSEVVYRKAAHEKDGYYLKFSCDEKLLEAKAT | ToxN/AbiQ |
| | LYSKKSTFQ | family toxin |
| | | [Lactococcus |
| | | lactis subsp. |
| | | lactis] |
| 9 | CBK89509.1 | Amino acid |
| | MIRNGFYIIKDRFFSDMSDPYLKGNKKQNRPHYYCFE | sequence |
| | DSNYNGIYWMIPLSSRIDKYKKIVSKRTGKGRNCDIIH | [Eubacterium] |
| | IVKLDDSHESAFLIQDMFPISDKYIEREYTIAGNHLRL | rectale] enzyme |
| | TSEHAAKEIEQKARKVLGMLKRGIKFTPTQPDIQKIYE | of CptIN |
| | RLQQDLPATHL | subfamily of |
| | | the type III |
| | | Toxin Antitoxin |
| | | Systems |
| 10 | CGCAAUUGCCGCAUUACAAGGGAAAUAACUUCGUA | RNA molecule |
| | CGUUGUUGUUAGCAU | comprising |
| | | cleavage site |
| | | for ToxN |
| | | comprises the |
| | | amino acid |
| | | sequence SEQ |
| | | ID No. 1 |
| 11 | AGGTCATATGGCGAAATTTTTCACAATATCA | Forward primer |
| | | for cloning |
| | | E.coli ToxN1 |
| | | SEQ ID NO. 3 |
| 12 | GGAAATAGACGATCTCGAGTCTAGAGCAT | Revers primer |
| | | for cloning |
| | | E.coli ToxN1 |
| | | SEQ ID NO. 3 |
| 13 | WP_208634907 | type III toxin- |
| | MKFFIVSDRY ISYLKKIDAK VPDNYNGKRP | antitoxin |
| | FIGIVVTVNG IEYVAPLSSPKPQLERINNS | system |
| | KPSVFKMFSR KDANDFLGVI NLNYMIPYLA | ToxN/AbiQ |
| | SEVTLLDVDN IQDHKYKNLLQKQHEYIKIN | family toxin |
| | KEEISSKATK LHDLVRNKKQ DHFVSISCDF | [Escherichia |
| | DALENALKSF QAT | albertii] (ET- |
| | | N5) |

1. A composition comprising isolated ToxN endoribonuclease or an enzymatic active fragment thereof, wherein
a concentration of a monovalent salt in the composition is ≤150 mM.

2. A sample comprising at least one polyribonucleotide and an isolated ToxN endoribonuclease or an enzymatic active fragment thereof, wherein
a concentration of a monovalent salt in the sample is about ≤150 mM.

3. A method of cleaving single stranded RNA molecules in a sample, wherein the method comprises the steps:
a. providing a sample, wherein the sample comprises at least one single stranded RNA molecule comprising a cleavage site for a ToxN endoribonuclease; and
b. contacting a ToxN endoribonuclease or an enzymatically active fragment thereof with the at least one RNA molecule in said sample under conditions which permit cleavage of at least a portion said RNA molecule present in the sample, and wherein concentration of a monovalent salt in the sample is about ≤150 mM.

4. The method according to claim 3, wherein said single stranded RNA molecule in step a) is a concatemer comprising multiple copies of precursors of either mRNA, siRNA, circular RNA, microRNA, or ribozyme; and wherein the concatemeric RNA molecule comprises a cleavage site for a ToxN endoribonuclease between each copy of the precursors of mRNA, siRNA, circular RNA, microRNA, or ribozyme.

5. A method for determining 5′-capping efficiency of an RNA molecule, wherein the method comprises the steps:
a. providing a sample comprising at least one single stranded mRNA molecule comprising a cleavage site for a ToxN endoribonuclease in the 5′UTR of said mRNA;
b. contacting said sample from step a) with a ToxN endoribonuclease under conditions that permit cleavage of at least a portion of said single stranded RNA molecules to produce at least one 5′ terminal RNA fragment and at least one 3′ terminal RNA fragment, wherein a concentration of a monovalent salt in the sample is about ≤150 mM; and
c. separating and detecting the RNA fragments from step b) and determining the presence of a 5′-capping modification at the 5′end of said 5′ terminal RNA fragments.

6. A method of RNA fingerprinting, wherein the method comprises the steps:
a. providing a sample comprising a at least one single stranded RNA molecule with unknown sequence;
b. contacting said sample from step a) with a ToxN endoribonuclease under conditions that permit cleavage of at least a portion of said RNA molecules thereby obtaining a plurality of RNA fragments, wherein a concentration of a monovalent salt in the sample is about ≤150 mM; and
c. separating and detecting the fragmented RNA molecule from step b, thereby obtaining a fingerprint of said RNA molecule with unknown sequence, and comparing the obtained fingerprint with fingerprints from RNA molecules with known sequence.

7. The method according to claim 5, wherein the separation and detection steps are selected from gel electrophoresis, capillary gel-electrophoresis (CGE), PAGE, high pressure liquid chromatography (HPLC), mass spectrometry (MS) or LC-MS, IP RP HPLC, and LC-UV.

8. The method according to claim 6, wherein the RNA molecule is selected from an mRNA, RNA virus, an immunogenic RNA molecule, a viroid, along non-coding RNA, and a ribozyme.

9. The composition according to claim 1, wherein the composition or sample is essentially without divalent metal cations wherein the divalent metal cations are Mg²⁺ or Mn²⁺.

10. The composition according to claim 1, wherein a concentration of divalent metal cations in the composition is about ≤3 mM, wherein the divalent metal cations are Mg²⁺ or Mn²⁺.

11. The composition according to claim 1, wherein the composition or sample comprises a ratio of concentration of a divalent metal cation to concentration of divalent ion chelating agent in the composition or sample providing that a concentration of free divalent metal cation present in the composition is about ≤3 mM, wherein the divalent metal cations are Mg²⁺ or Mn²⁺.

12. The composition according to claim 1, wherein the composition comprises a concentration of a divalent ion chelating agent of about ≤10 mM.

13. The composition according to claim 1, wherein the isolated ToxN endoribonuclease comprises an amino acid sequence of SEQ ID NO: 1 or an enzymatically active fragment thereof, or a ToxN endoribonuclease comprising an amino acid sequence which is at least 30% identical to SEQ ID NO: 1.

14. The composition according to claim 1, wherein the isolated ToxN endoribonuclease is not in a complex with ToxI RNA.

15. A kit comprising:
a. the composition according to claim 1; and
b. a second composition comprising a second enzyme selecting from a group consisting of an RNA polymerase, an RNA ligase for ligating single stranded RNA molecules, a pyrophosphatase, a phosphatase, a kinase, another nucleic acid or ribonucleic acid modifying enzyme and at least one further ToxN endoribonuclease with a different recognition site from the ToxN endoribonuclease of a).

16. The composition of claim 1, wherein the concentration of the monovalent salt in the composition is ≤100 mM.

17. The composition of claim 1, wherein the monovalent salt is an alkali metal salt.

18. The sample of claim 2, wherein the concentration of the monovalent salt in the composition is ≤100 mM.

19. The sample of claim 2, wherein the monovalent salt is an alkali metal salt.

20. The method of claim 3, wherein the concentration of the monovalent salt in the composition is ≤100 mM.

21. The method of claim 3, wherein the monovalent salt is an alkali metal salt.


SEQUENCE LISTING (13 sequences)

SEQ ID NO: 1 - protein, 176 aa, Escherichia coli
MAKFFTISSSYIKYLKDFDDKVPNSEDPTYNNPKAFIGIVLEIEGHKYLAPLTSPKAWHANVKESSPAFFKLHENGVPDNQLGLINLKFMIPIIEAEVSLLDLDSMPDTPYKRMLYKQLQFIRVNEDKISEKSKLLRNLALQGRMQGTCDFAVLEEKYQHFGKKPEDMEIDDLESR

SEQ ID NO: 2 - protein, 187 aa, Escherichia coli
MNHKVHHHHHHMAKFFTISSSYIKYLKDFDDKVPNSEDPTYNNPKAFIGIVLEIEGHKYLAPLTSPKAWHANVKESSPAFFKLHENGVPDNQLGLINLKFMIPIIEAEVSLLDLDSMPDTPYKRMLYKQLQFIRVNEDKISEKSKLLRNLALQGRMQGTCDFAVLEEKYQHFGKKPEDMEIDDLESR

SEQ ID NO: 3 - DNA, 516 bp, Escherichia coli
  source 1..516: mol_type = genomic DNA
atgaaatttttcacaatatcaagcagttacataaaatacctgaaggactttgatgacaaagttcccaatagcgaagatcctacatacaacaatcctaaggctttcattggcatagtactagagattgaaggacataaatatttagcacctttaacatcgccaaaagcatggcatgctaatgtaaaagagtcatcacccgccttcttcaaacttcatgaaaacggtgtccctgataatcagcttggattgattaatctgaaatttatgattccaataatcgaagctgaagtgtctttacttgatctggatagcatgcctgatacgccttacaaaagaatgctatataaacagttacaatttattcgtgtaaatgaagataaaatatctgaaaaatcaaaactattaagaaaccttgccttgcaaggcagaatgcaaggaacatgcgactttgccgtgttagaagaaaaatatcaacatttcggtaaaaaacctgaagacatggaaatagacgattaa

SEQ ID NO: 4 - DNA, 166 bp, Escherichia coli
  source 1..166: mol_type = genomic DNA
aggtgatttgctacctttaagtgcagctagaaatttaggtgatttgctacctttaagtgcagctagaaatttaggtgatttgctacctttaagtgcagctagaaattcaggtgatttgctacctttaagtgcagctagaaatttaggtcatttactaccttaaagt

SEQ ID NO: 5 - RNA, 36 nt, Escherichia coli
  source 1..36: mol_type = transcribed RNA
attcaggtgatttgctacctttaagtgcagctagaa

SEQ ID NO: 6 - protein, 171 aa, Pectobacterium atrosepticum
MKFYTISSKYIEYLKEFDDKVPNSEDPTYQNPKAFIGIVLEIQGHKYLAPLTSPKKWHNNVKESSLSCFKLHENGVPENQLGLINLKFMIPIIEAEVSLLDLGNMPNTPYKRMLYKQLQFIRANSDKIASKSDTLRNLVLQGKMQGTCNFSLLEEKYRDFGKEAEDTEEGE

SEQ ID NO: 7 - protein, 194 aa, Bacillus thuringiensis
MTNKDNPKFHTISTEYIDYLREADSKVPFNKDEQHSRPYVGVLEKINGHDYFVPLTSRNDKNFNSQVSVKLFDNDEKRIGVLLVNNMIPVPEKECKEIDIAEKTAADPQYGNLMLKQYLFLKENMDRVTNKVEKVYKDVTVQGKPSHKQKFLKGVCCDFPKLEEKCQEYKERDQAKERDKARRIAYMRQMGRER

SEQ ID NO: 8 - protein, 183 aa, Lactococcus lactis
MSSFFYKEILRMTLRFFTVTDEYIAYLRKFESKVHYQYENNASTYVGVVLKKNDFNYFIPLSSYKKGNPEKDKAMKKRSRIVTRLFEIGNINNPLGYLLHHNMIPVPDSELIPLPLDLKKPKHKMMQKQLIYMKSISEKIENKSEVVYRKAAHEKDGYYLKFSCDFKLLEAKATLYSKKSTFQ

SEQ ID NO: 9 - protein, 162 aa, Eubacterium rectale
MIRNGFYIIKDRFFSDMSDPYLKGNKKQNRPHYYCFEDSNYNGIYWMIPLSSRIDKYKKIVSKRTGKGRNCDIIHIVKLDDSHESAFLIQDMFPISDKYIEREYTIAGNHLRLTSEHAAKEIEQKARKVLGMLKRGIKFTPTQPDIQKIYERLQQDLPATHL

SEQ ID NO: 10 - RNA, 50 nt, synthetic construct
  source 1..50: mol_type = other RNA
cgcaattgccgcattacaagggaaataacttcgtacgttgttgttagcat

SEQ ID NO: 11 - DNA, 31 bp, synthetic construct
  source 1..31: mol_type = other DNA
aggtcatatggcgaaatttttcacaatatca

SEQ ID NO: 12 - DNA, 29 bp, synthetic construct
  source 1..29: mol_type = other DNA
ggaaatagacgatctcgagtctagagcat

SEQ ID NO: 13 - protein, 163 aa, Escherichia albertii
MKFFIVSDRYISYLKKIDAKVPDNYNGKRPFIGIVVTVNGIEYVAPLSSPKPQLERINNSKPSVFKMFSRKDANDFLGVINLNYMIPYLASEVTLLDVDNIQDHKYKNLLQKQHEYIKINKEEISSKATKLHDLVRNKKQDHFVSISCDFDALENALKSFQAT